SEQUENCE LISTING (44 sequences)

SEQ ID NO: 1 - protein, 5 aa, Homo sapiens
YGGFL

SEQ ID NO: 2 - protein, 5 aa, Homo sapiens
YGGFM

SEQ ID NO: 3 - protein, 17 aa, Homo sapiens
YGGFLRRIRPKLKWDNQ

SEQ ID NO: 4 - protein, 13 aa, Homo sapiens
YGGFLRRIRPKLK

SEQ ID NO: 5 - protein, 8 aa, Homo sapiens
YGGFLRRI

SEQ ID NO: 6 - protein, 13 aa, Homo sapiens
YGGFLRRNFLVVT

SEQ ID NO: 7 - protein, 4 aa, Artificial Sequence
  Peptide has C-terminal Amidation
  SITE (2)..(2): Xaa refers to D-Alanine
  MOD_RES (4)..(4): F is amidated with -NH2, e.g., F is Phe-NH2
YAGF

SEQ ID NO: 8 - protein, 4 aa, Artificial Sequence
  Peptide has C-terminal amidation
  MISC_FEATURE (2)..(2): Ala is D-Alanine
  MOD_RES (4)..(4): F is amidated with N-phenyl-N-piperidin-4-ylpropionamide
YAGF

SEQ ID NO: 9 - protein, 4 aa, Artificial Sequence
  peptide has C-terminal amidation
  MISC_FEATURE (1)..(1): Xaa is 2,6-dimethyltyrosine
  MISC_FEATURE (2)..(2): Ala is D-Alanine
  MOD_RES (4)..(4): Phe is amidated with N-phenyl-N-piperidin-4-ylpropionamide
XAGF

SEQ ID NO: 10 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with N-phenyl-N-piperidin-4-ylpropionamide
  MOD_RES (4)..(4): Phe halogenated with Fluorine
XXGF

SEQ ID NO: 11 - protein, 4 aa, Artificial Sequence
  Peptide has C-terminal amidation
  MISC_FEATURE (2)..(2): Xaa is D-Norleucine
  MOD_RES (4)..(4): Phe is amidated with -NH2
  MOD_RES (4)..(4): Phe is halogenated with fluorine
YXGF

SEQ ID NO: 12 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is N-Norleucine
  MOD_RES (4)..(4): Phe is amidated with -NH2
  MOD_RES (4)..(4): Phe is halogenated with Fluorine
XXGF

SEQ ID NO: 13 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with -NH2
XXGF

SEQ ID NO: 14 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is amidated with N-phenyl-N-piperidin-4-ylpropionamide
XXGF

SEQ ID NO: 15 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Cl
  MOD_RES (4)..(4): Phe is amidated with N-phenyl-N-piperidin-4-ylpropionamide
XXGF

SEQ ID NO: 16 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Tic (D-tetrahydroisoquinoline-3-carboxylic acid)
  MISC_FEATURE (3)..(3): Xaa is absent
  MOD_RES (4)..(4): Phe is amidated with N-phenyl-N-piperidin-4-ylpropionamide
XXXF

SEQ ID NO: 17 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Cl
  MOD_RES (4)..(4): Phe is amidated with -NH2
XXGF

SEQ ID NO: 18 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MOD_RES (4)..(4): Phe is halogenated with Br
  MOD_RES (4)..(4): Phe is amidated with N-phenyl-N-piperidin-4-ylpropionamide
XXGF

SEQ ID NO: 19 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Norleucine
  MISC_FEATURE (3)..(3): Xaa is homocysteine
  MOD_RES (4)..(4): Phe is halogenated with fluorine
  MOD_RES (4)..(4): Phe is amidated with N-phenyl-N-piperidin-4-ylpropionamide
XXXF

SEQ ID NO: 20 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is homocysteine
  MOD_RES (4)..(4): Phe is halogenated with fluorine
  MOD_RES (4)..(4): Phe is amidated with N-phenyl-N-piperidin-4-ylpropionamide
XXGF

SEQ ID NO: 21 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Arg is D-Arginine
  MOD_RES (4)..(4): Gly is amidated with N-phenyl-N-piperidin-4-ylpropionamide
XRFG

SEQ ID NO: 22 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Arg is D-Arginine
  MISC_FEATURE (3)..(3): Xaa is 1-naphthylalanine
  MOD_RES (4)..(4): Gly is amdiated with N-phenyl-N-piperidin-4-ylpropionamide
XRXG

SEQ ID NO: 23 - protein, 4 aa, Artificial Sequence
  Peptide has a C-terminal amidation
  MISC_FEATURE (1)..(1): Xaa is 2,6-dimethyltyrosine
  MISC_FEATURE (2)..(2): Arg is D-Arginine
  MOD_RES (4)..(4): Lys is amidated with N-phenyl-N-piperidin-4-ylpropionamide
XRFK

SEQ ID NO: 24 - protein, 4 aa, Artificial Sequence
  Peptide has a C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is 1-naphthylalanine
  MOD_RES (4)..(4): Lys is amidated with N-phenyl-N-piperidin-4-ylpropionamide
XRXK

SEQ ID NO: 25 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is absent
  MISC_FEATURE (4)..(4): Xaa is 1-naphthylalanine
  MOD_RES (4)..(4): 1-naphthylalanine is amidated with N-phenyl-N-piperidin-4-ylpropionamide
XRXX

SEQ ID NO: 26 - protein, 4 aa, Artificial Sequence
  Peptide has C-terminal amidation
  MISC_FEATURE (1)..(1): Xaa is 2,6-dimethyltyrosine
  MISC_FEATURE (2)..(2): Xaa is D-Arginine
  MISC_FEATURE (2)..(2): Arg is D-Arginine
  MISC_FEATURE (3)..(3): Xaa is absent
  MOD_RES (4)..(4): Phe is halogenated with Cl
  MOD_RES (4)..(4): Phe is amidated with N-phenyl-N-piperidin-4-ylpropionamide
XRXF

SEQ ID NO: 27 - protein, 4 aa, Artificial Sequence
  Peptide has C-terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPWF

SEQ ID NO: 28 - protein, 4 aa, Artificial Sequence
  Peptide has C_terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPFF

SEQ ID NO: 29 - protein, 4 aa, Artificial Sequence
  Peptide has C-terminal amidation
  MISC_FEATURE (1)..(1): Xaa is 2,6-dimethyltyrosine
  MOD_RES (3)..(3): Phe is halogenated with Cl
  MOD_RES (4)..(4): Phe is halogenated with Cl
  MOD_RES (4)..(4): Phe is amidated with N-phenyl-N-piperidin-4-ylpropionamide
XPFF

SEQ ID NO: 30 - protein, 4 aa, Artificial Sequence
  Peptide has C-terminal amidation
  MISC_FEATURE (1)..(1): Xaa is 2,6-dimethyltyrosine
  MOD_RES (4)..(4): Phe is halogenated with Cl
  MOD_RES (4)..(4): Phe is amidated with N-phenyl-N-piperidin-4-ylpropionamide
XPGF

SEQ ID NO: 31 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 32 - protein, 4 aa, Artificial Sequence
  Peptide has a C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 33 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 34 - protein, 4 aa, Artificial Sequence
  Peptide has C-terminal amidation
  MISC_FEATURE (2)..(2): Xaa is D-Alanine
  MISC_FEATURE (2)..(2): Ala is D-Alanine
  MOD_RES (4)..(4): Phe is amidated with a 4-anilidopiperidine moiety
YAGF

SEQ ID NO: 35 - protein, 4 aa, Homo sapiens
  MOD_RES (4)..(4): Phe is amidated with -NH2
YPWF

SEQ ID NO: 36 - protein, 4 aa, Artificial Sequence
  Dermorphin analog
  MISC_FEATURE (2)..(2): Xaa is D-Arginine
  MISC_FEATURE (2)..(2): Arg is D-Arginine
  MOD_RES (4)..(4): Lys is amidated with -NH2
YRFK

SEQ ID NO: 37 - protein, 4 aa, Artificial Sequence
  Peptide has C-terminal amidation
  MOD_RES (4)..(4): PHE is amidated with N-phenyl-N-piperidin-4-ylpropionamide
YGGF

SEQ ID NO: 38 - protein, 5 aa, Artificial Sequence
  Peptide has C-terminal amidation
  MISC_FEATURE (1)..(1): Xaa is 2,6-dimethyltyrosine
  MISC_FEATURE (2)..(2): D-Norleucine (D-Nle)
  MOD_RES (4)..(4): Phe is halogenated with F
  MOD_RES (4)..(4): Phe is amidated with N-phenyl-N-piperidin-4-ylpropionamide
XXGFL

SEQ ID NO: 39 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid (Tic)
  MOD_RES (4)..(4): Phe is amidated with N-phenyl-N-piperidin-4-ylpropionamide
XXGF

SEQ ID NO: 40 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid
  MOD_RES (4)..(4): Phe is halogenated with F
  MOD_RES (4)..(4): Phe is amidated with N-phenyl-N-piperidin-4-ylpropionamide
XXGF

SEQ ID NO: 41 - protein, 4 aa, Artificial Sequence
  Peptide has C-terminal amidation
  MISC_FEATURE (1)..(1): 2,6-dimethyltyrosine
  MISC_FEATURE (2)..(2): tetrahydroisoquinoline-3-carboxylic acid
  MOD_RES (4)..(4): F is halogenated with Cl
  MOD_RES (4)..(4): F is amidated with N-phenyl-N-piperidin-4-ylpropionamide
XXGF

SEQ ID NO: 42 - protein, 4 aa, Artificial Sequence
  Peptide has C-terminal amidation
  MISC_FEATURE (1)..(1): 2,6-dimethyltyrosine
  MISC_FEATURE (2)..(2): D-Arginine
  MOD_RES (3)..(3): Phe is halogenated with Cl
  MOD_RES (4)..(4): L is amidated with N-phenyl-N-piperidin-4-ylpropionamide
XXFL

SEQ ID NO: 43 - protein, 4 aa, Artificial Sequence
  Peptide has C-terminal amidation
  MISC_FEATURE (1)..(1): 2,6-dimethyltyrosine
  MISC_FEATURE (2)..(2): D-Arginine
  MISC_FEATURE (3)..(3): Phe is halogenated with F
  MISC_FEATURE (4)..(4): L is amidated with N-phenyl-N-piperidin-4-ylpropionamide
XXFL

SEQ ID NO: 44 - protein, 4 aa, Artificial Sequence
  Peptide has C-terminal amidation
  MISC_FEATURE (1)..(1): D-Phenylalanine
  MISC_FEATURE (2)..(2): D-Phenylalanine
  MISC_FEATURE (3)..(3): D-Norleucine
  MISC_FEATURE (4)..(4): D-Lysine
  MOD_RES (4)..(4): D-Lys is amidated with N-phenyl-N-piperidin-4-ylpropionamide
XXXX

(19) United States
(12) Patent Application Publication
(10) Pub. No.: US 2021/0196782 A1
(43) Pub. Date: Jul. 1, 2021

(54) MULTIFUNCTIONAL OPIOID RECEPTOR LIGANDS AND METHODS OF TREATING PAIN

(71) Applicant: ARIZONA BOARD OF REGENTS ON BEHALF OF THE UNIVERSITY OF ARIZONA, Tucson, AZ (US)

(72) Inventors: Yeon Sun Lee, Tucson, AZ (US); Victor J. Hruby, Tucson, AZ (US); Frank Porreca, Tucson, AZ (US)

(21) Appl. No.: 17/197,980

(22) Filed: Mar. 10, 2021

Related U.S. Application Data
(63) Continuation-in-part of application No. 15/820,133, filed on Nov. 21, 2017.
Application No. 15/820,133 is a continuation-in-part of application No. PCT/US16/33529, filed on May 20, 2016.
(60) Provisional application No. 62/165,063, filed on May 21, 2015.
(60) Provisional application No. 62/476,980, filed on Mar. 27, 2017.

(51) Int. Cl. A61K 38/07

(52) U.S. Cl. CPC A61K 38/07

(57) ABSTRACT

Opioid receptor ligands (ORLs) that are multifunctional having agonist activity at mu opioid receptor (MOR), agonist activity at delta opioid receptor (DOR), and antagonist (or partial agonist) activity at kappa opioid receptor (KOR) are for the treatment of pain. The ORLs comprise peptide portions that are analogs derived from enkephalins, endomorphins, or [DArg², Lys⁴]-dermorphine (DALDA), as well as tail portions that comprise a lipophilic molecule such as a 4-anilidopiperidine moiety.

### CROSS REFERENCE

This application is a continuation-in-part and claims benefit of U.S. patent application Ser. No. 15/820,133 filed Nov. 21, 2017, which is a continuation-in-part and claims benefit of PCT/US16/33529 filed May 20, 2016, which claims benefit of U.S. Provisional Patent Application No. 62/165,063 filed May 21, 2015; and U.S. Ser. No. 15/820,133 also claims benefit of U.S. Provisional Patent Application No. 62/476,980 filed Mar. 27, 2017, the specification(s) of which are incorporated herein in their entirety by reference.

### GOVERNMENT SUPPORT

This invention was made with government support under Grant No. P01 DA006284, awarded by National Institutes of Health. The government has certain rights in the invention.

### REFERENCE TO A SEQUENCE LISTING

Applicant asserts that the paper copy of the Sequence Listing is identical to the Sequence Listing in computer readable form found on the accompanying computer file, entitled UNIA_15_13_PCT_ClP2_Sequence_Listing_ST25. The content of the sequence listing is incorporated herein by reference in its entirety.

### FIELD OF THE INVENTION

The present invention relates to ligands for mu, delta, and kappa opioid receptors, more particularly to multifunctional opioid peptides that function as mu opioid receptor (MOR), delta opioid receptor (DOR) agonists, and kappa opioid receptor (KOR) antagonists (or partial agonists). The present invention also relates to treating pain or other conditions using the multifunctional opioid peptides herein. The present invention also relates to pharmacophores for modifying C-terminal regions of opioid peptides (e.g., enkephalins, dermorphines, endomorphines, etc.) for conferring particular KOR activity.

### BACKGROUND OF THE INVENTION

Opioids are commonly used in the treatment of severe pain. Opioids have analgesic activity through their interaction with the opioid receptors (e.g., mu (μ) opioid receptor (MOR), delta (δ) opioid receptor (DOR), kappa (κ) opioid receptor (KOR)), mostly with MOR. However, the clinical use of opioids is limited by associated side effects such as respiratory depression, constipation, development of tolerance, and addiction. Indeed, chronic pain and subsequent chronic administration of a MOR agonist can lead to KOR activation, which results in undesirable adverse and addictive behaviors. For this reason, a KOR antagonist (or partial agonist) could be used to reduce such undesirable effects of chronic MOR activation.

Inventors have surprisingly discovered opioid peptides, e.g., opioid receptor ligands (ORLs) that are multifunctional, e.g., acting as MOR agonists, DOR agonists, and KOR antagonists (or partial agonists). Without wishing to limit the present invention to any theory or mechanism, it is believed that this MOR/DOR agonist with KOR antagonist/partial agonist activity encompassed by a single molecule may be better and/or more effective than using co-administration of two or more molecules to achieve MOR/DOR agonist and KOR antagonist/partial agonist activity.

In some embodiments, the multifunctional ORLs may comprise peptide analogs derived from enkephalins. Enkephalins are pentapeptides (peptides containing 5 amino acids) that are endogenous ligands of the opioid receptors (e.g., MOR, and DOR). There are two known forms of enkephalins: leucine-containing enkephalin (Leu-Enk, or YGGFL (SEQ ID NO: 1)) and methionine-containing enkephalin (Met-Enk, or YGGFM (SEQ ID NO: 2)). In some embodiments, the multifunctional ORLs comprise peptide analogs derived from endomorphin-1 (EM-1), endomorphin-2 (EM-2) or other opioid ligands such as DALDA ([DArg², Lys⁴]-dermorphin), FE20066, etc.

In some embodiments, the ORLs comprise a 4-anilidopiperidine moiety, e.g., fentanyl analog, an analog of a 4-anilidopiperidine, etc., e.g., N-phenyl-N-piperidin-4-ylpropionamide (Ppp).

The present invention also provides C-terminal modifications (pharmacophores) that confer KOR antagonist activity to opioid peptides. The present invention also provides modifications, such as halogenation of a phenylalanine (Phe) and a Ppp moiety, that confer KOR antagonist or partial agonist activity. Those kappa activities may help reduce KOR- or MOR-related side effects. For example, modifications of opioid ligands (such as DALDA, EM-1, EM-2, and FE20066) with pharmacophores (e.g., the C-terminal modifications of the aforementioned molecules) may generate the similar KOR activity.

For example, the present invention provides ORLs with a Ppp tail, wherein the Ppp comprises an R group (e.g., a halogen).

### SUMMARY OF THE INVENTION

The present invention features multifunctional opioid receptor ligands (ORLs) and methods of use of said multifunctional ORLs.

In certain embodiments, the ORLs herein have agonist activity at mu opioid receptor (MOR), agonist activity at delta opioid receptor (DOR), and antagonist activity at kappa opioid receptor (KOR). In certain embodiments, the ORLs herein have agonist activity at mu opioid receptor (MOR), agonist activity at delta opioid receptor (DOR), and partial agonist activity at kappa opioid receptor (KOR).

The present invention provides multifunctional opioid receptor ligands (ORLs) according to Formula 1: Aaa-Bbb-Ccc-Ddd(X)-Eee.

In some embodiments, Aaa is selected from 2′-6′-dimethyltyrosine (Dmt), Tyrosine (Tyr), β-methyl-2′,6′-dimethyl tyrosine (Tmt), Phe, 2′,6′-dimethylphenylalanine (Dmp), and 2-methyl-3-(2,6-dimethyl-4-hydroxyphenyl)propanoic acid (Mdp); Bbb is selected from D-Alanine (DAa), Alanine (Ala), D-Norleucine (DNle), Norleucine (Ne), Proline (Pro), Pro, D-Arginine (DArg), Arg, D-Tetrahydroisoquinoline-3-carboxylic acid (DTic), and Tic; Ccc is selected from Glycine (Gly), Phenylalanine(X) (Phe(X)), tryptophan (Trp), and naphthylalanine (Nal) or is absent; Ddd(X) is Gly, Phe(X), Trp, naphthylalanine (Nal), or Lysine (Lys); and Eee comprises N-phenyl-N-piperidin-4-ylpropionamide-R (Ppp(R)); wherein X and R both comprise a hydrogen or a halogen. In some embodiments, X is selected from H, F, Cl, and Br. In some embodiments, X is selected from H, F, and Cl. In certain embodiments, R is selected from H, 3-Cl, 4-Cl, 3-F, 4-F, and 2,4-diCl.

In some embodiments, Aaa is selected from Dmt, Tyr, Tmt, Phe, Dmp, and Mdp; Bbb is selected from DAla, Nle, and DTic; Ccc is selected from Gly, Phe(X), Trp, and Nal or is absent; Ddd(X) is Gly, Phe(X), Trp, Nal, or Lys; and Eee comprises Ppp(R); wherein X and R both comprise a hydrogen or a halogen. In some embodiments, X is selected from H, F, Cl, and Br. In some embodiments, X is selected from H. F, and Cl. In certain embodiments, R is selected from 3-Cl, 4-Cl, 3-F, 4-F, and 2,4-diCl. In some embodiments, the ORL is SEQ ID NO: 8, SEQ ID NO: 9, SEQ ID NO: 10, SEQ ID NO; 14, SEQ ID NO: 15, SEQ ID NO: 16.

In some embodiments, Aaa is selected from Tmt, Phe, Dmp, and Mdp; Bbb is selected from DAla, Ala, Ne, Ne, Pro, Pro, DArg, Arg, DTic, and Tic; Ccc is selected from Gly, Phe(X), Trp, and Nal or is absent; Ddd(X) is Gly, Phe(X), Trp, Nal, or Lys; and Eee comprises Ppp(R): wherein X and R both comprise a hydrogen or halogen. In some embodiments, X is selected from H, F, Cl, and Br. In some embodiments, X is selected from H, F, and Cl. In certain embodiments, R is selected from 3-Cl, 4-Cl, 3-F, 4-F, and 2,4-diCl.

In some embodiments, Aaa is selected from Dmt, Tyr, Tmt, Phe, Dmp, and Mdp; Bbb is selected from Ala, Ne, Pro, Arg, Tic; Ccc is selected from Gly, Phe(X), Trp, and Nal or is absent, wherein X is a halogen; Ddd(X) is Gly, Phe(X), Trp, Nal, or Lys, wherein X is a halogen; and Eee is a 4-anilidopiperidine moiety (e.g., Ppp). In some embodiments, Ppp comprises Ppp(R), wherein R comprises a hydrogen or halogen. In some embodiments, R is selected from 3-Cl, 4-Cl, 3-F, 4-F, and 2,4-diCl. In some embodiments, X is selected from H, F, Cl, and Br.

The present invention also provides multifunctional ORLs according to Formula 2: Aaa-Bbb-Ccc-Ddd(X)-Yyy(n)-Eee.

In some embodiments, Aaa is selected from Dmt, Tyr, Tmt, Phe, Dmp, and Mdp; Bbb is selected from DAla, Ne, Pro, and DArg, Tic, DTic; Ccc is selected from Gly, Phe(X), Trp, and Nal or is absent, wherein X is a hydrogen or a halogen; Ddd(X) is Gly, Phe(X), Trp, Nal, or Lys, wherein X is a hydrogen or a halogen; Yyy is selected from one or a combination of Leu, Arg, Met, Lys, or lie; and Eee is a 4-anilidopiperidine moiety (e.g., Ppp).

In some embodiments, Ppp comprises Ppp(R), wherein R comprises a hydrogen or a halogen. In some embodiments, R is selected from 3-Cl, 4-Cl, 3-F, 4-F, and 2,4-diCl. In some embodiments, X is selected from H, F, Cl, and Br. In some embodiments, n=1. In some embodiments, n=2. In some embodiments, n=3. In some embodiments, n=4. In some embodiments, n=5. In some embodiments, n=6. In some embodiments, n=7. In some embodiments, n=8. In some embodiments, n is 8 or more, e.g., 9, 10, 11, 12, 13, etc.

The present invention also provides multifunctional ORLs according to Formula 3: Aaa-DArg-Ccc-Ddd-Eee.

In some embodiments, Aaa is selected from Tyr or Dmt; Ccc is selected from Phe, Phe(X), or 1Nal; Ddd is selected from Lys, Gly or is absent; Eee is a 4-anilidopiperidine moiety; and X is selected from F, C, or Br. In some embodiments, the 4-anilidopiperidine moiety comprises Ppp. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is SEQ ID NO: 21, SEQ ID NO: 22, SEQ ID NO: 23, SEQ ID NO; 24, SEQ ID NO: 25, SEQ ID NO: 26, SEQ ID NO: 42, or SEQ ID NO: 43.

The present invention also provides multifunctional ORLs according to Formula 4: Aaa-Pro-Ccc-Phe(X)-Eee.

In some embodiments, Aaa is selected from Tyr or Dmt; Ccc is selected from Trp, Phe, Gly, or Phe(X); Eee is a 4-anilidopiperidine moiety (e.g., Ppp), and X is selected from F, Cl, or Br. In some embodiments, Ppp comprises Ppp(R), wherein R comprises a halogen. In some embodiments, R is selected from 3-Cl, 4-Cl, 3-F, 4-F, and 2,4-diCl. In some embodiments, the ORL is selected from SEQ ID NO: 27, SEQ ID NO: 28, SEQ ID NO: 29, or SEQ ID NO: 30.

The present invention also provides multifunctional opioid receptor ligands (ORLs) according to Formula 5: DPhe-DPhe-DNle-Ddd-Eee.

In some embodiments, Ddd is selected from DArg or DLys, and Eee is a 4-anilidopiperidine moiety (e.g., Ppp). For example, in some embodiments, Ddd is DArg and Eee is Ppp. In some embodiments, Ddd is DLys and Eee is Ppp. In some embodiments, Ppp comprises Ppp(R), wherein R comprises a halogen. For example, in some embodiments, Ddd is DArg and Eee is Ppp(R), wherein R comprises a halogen (e.g., Cl, F, Br). In some embodiments, Ddd is DLys and Eee is Ppp(R), wherein R comprises a halogen (e.g., Cl, F, Br). In some embodiments, R is selected from 3-Cl, 4-Cl, 3-F, 4-F, and 2,4-diCl. In some embodiments, the ORL is according to SEQ ID NO: 44.

The present invention also provides an opioid receptor ligand dimer according to SEQ ID NO: 19. The present invention also provides an opioid receptor ligand dimer according to SEQ ID NO: 20.

The present invention also provides methods of reducing pain, e.g., reducing pain in a subject in need of a KOR antagonist or KOR partial agonist. In some embodiments, the method comprises identifying a subject in need of a kappa opioid receptor (KOR) antagonist or partial agonist, and introducing to the subject a multifunctional ORL according to the present invention, wherein the ORL is effective for reducing pain.

The present invention also features methods of blocking kappa opioid receptor. In some embodiments, the method comprises introducing to the KOR a multifunctional ORL according to the present invention.

The present invention also features methods of blocking KOR, activating MOR, and activating DOR in a subject. In some embodiments, the method comprises introducing to the subject a multifunctional ORL according to the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The patent application or application file contains at least one drawing executed in color. Copies of this patent or patent application publication with color drawing(s) will be provided by the Office upon request and payment of the necessary fee.

The features and advantages of the present invention will become apparent from a consideration of the following detailed description presented in connection with the accompanying drawings in which:

FIG. 1 shows the well-known structure-activity relationship (SAR) results of Dynorphin A (Dyn A (SEQ ID NO: 3)) and Enkephalins for opioid activities. Peptides tested include Dyn A (SEQ ID NO: 3), an endogenous KOR ligand, a peptide containing the first 13 amino acids of Dyn A (Dyn A 1-13 (SEQ ID NO: 4)), a peptide containing the first 8 amino acids of Dyn A (Dyn A 1-8 (SEQ ID NO: 5)), Dyn B (SEQ ID NO: 6), Leu-Enk (SEQ ID NO: 1), and Met-Enk (SEQ ID NO: 2).

FIG. 2 shows non-limiting examples of anilidopiperidine analogs as tails of the ORLs of the present invention.

FIG. 3 shows GTPγS activity of LYS739 (SEQ ID NO: 10), U50,488, and Naloxone at KOR. U50,488 is known to have agonist activity at KOR. Naloxone is known to have antagonist activity at KOR. LYS739 (SEQ ID NO: 10) appears to have partial agonist/antagonist activities at KOR. LYS744 (SEQ ID NO: 15) appears to have full antagonist activity at KOR.

FIG. 4A shows [³⁵S]GTPγS assays: MOR (left) and DOR (right) antagonist modes. LYS739 (SEQ ID NO: 10), LYS744 (SEQ ID NO: 15), and MR115 (SEQ ID NO: 28) do not possess antagonist activity at MOR and DOR.

FIG. 4B shows [³⁵S]GTPγS assays: KOR agonist (left column) and antagonist (right column) modes. LYS540 (SEQ ID NO: 9), LYS644 (SEQ ID NO: 14), and MR121 (SEQ ID NO: 126) are partial agonist/antagonist at KOR. CYF132 (SEQ ID NO: 13), and MR107 (SEQ ID NO: 12) are observed as a partial agonist at KOR. MR115 (SEQ ID NO: 28) is observed as a partial antagonist at KOR.

FIG. 5A and FIG. 5B show the effects of fentanyl analogs, LYS436 (SEQ ID NO: 8), LYS739 (SEQ ID NO: 10) and LYS416 (SEQ ID NO: 37) and biphalin on H/A and reoxygenation challenge.

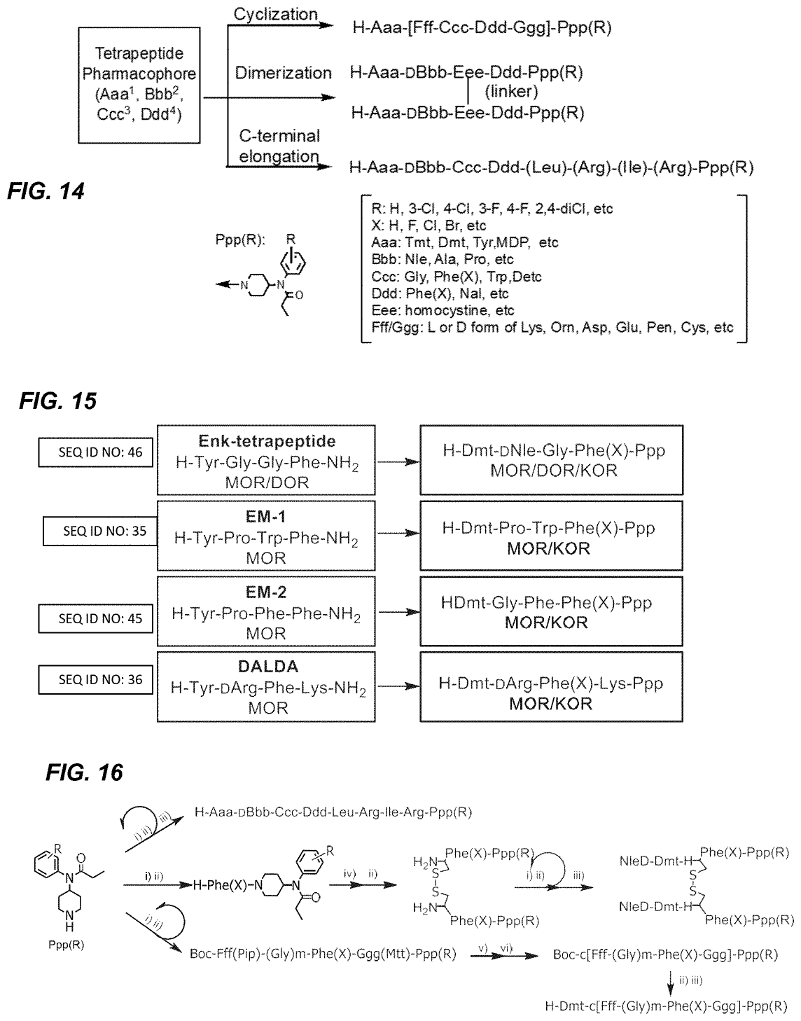

For both the graphs (FIGS. 5A and 5B); ‘*’ compared to no drug treated group; ‘#’ compared to biphalin treated group; *p<0.05, ***p<0.001, ****p<0.0001; #p<0.05, ##p<0.01; data from 3 to 4 independent primary neuron isolations with 2-3 replicates treatment per isolation. Compared to normoxic and 0.1% triton X, all experimental groups were significantly different (p<0.0001). A) MTT assay: Effect of fentanyl analogs LYS436, LYS739 and LYS416 and biphalin on 3 hr H/A ad 24 hr reoxygenation. Compared to no drug treated group, LYS436 (p<0.0001), LYS739 (p<0.0001), LYS416 (p<0.0001), biphalin (p<0.001) and fentanyl (p<0.05) significantly increased neuronal survival. Again, compared to biphalin, LYS739 (p<0.01) and LYS416 (p<0.05) showed better neuroprotection in terms of neuronal cell survival. LYS436 (p<0.05), LYS739 (p<0.0001) and LYS416 (p<0.001) demonstrated better neuronal survival compared to fentanyl alone. NTX reversed the effect of LYS436, LYS739, LYS416 and biphalin. B) LDH assay: Relative neuronal death in terms of LDH production was assessed upon 3 hr H/A and 24 hr reoxygenation. Fentanyl analogs LYS436 (p<0.001), LYS739 (p<0.0001) and LYS416 (p<0.0001) and biphalin (p<0.001) and fentanyl (p<0.05) significantly decreased neuronal cell death compared to no drug treated group. LYS739 (p<0.05) significantly decreased neuronal cell death in comparison to biphalin. LYS739 (p<0.001) and LYS416 (p<0.01) showed better neuroprotection compared to fentanyl alone. NTX reversed the effect of LYS436, LYS739, LYS416 and biphalin.

FIG. 6A and FIG. 6B shows the effects of fentanyl analogs, LYS436 (SEQ ID NO: 8), LYS739 (SEQ ID NO: 10) and LYS416 (SEQ ID NO: 37) and biphalin on NMDA challenge. For both the graphs; ‘*’ compared to no drug treated group; ‘#’ compared to biphalin treated group; *p<0.05, **p<0.01 ***p<0.001, ****p<0.0001; #p<0.05, ##p<0.01; data from 3 to 4 independent primary neuron isolations with 2-3 replicates treatment per isolation. All experimental groups were significantly different (p<0.0001) compared to normoxia and 0.1% tritonX. A) MTT assay: effects of fentanyl analogs and biphalin (10 nM) on primary cortical neurons with NMDA (50 uM) exposure for 3 hr assessed by relative neuronal survival. LYS436 (p<0.0001), LYS739 (p<0.0001), LYS416 (p<0.001), biphalin (p<0.01) and fentanyl (p<0.05) significantly improved relative neuronal survival compared to no drug treated group. The Effects of LYS739 (p<0.01) and LYS436 (p<0.05) were significantly better than biphalin. LYS436 (p<0.01) and LYS739 (p<0.001) also increased neuronal survival when compared to fentanyl alone. NTX reversed the effect of LYS436, LYS739, LYS416 and biphalin. B) LDH assay: effects of fentanyl analogs and biphalin (10 nM) on primary cortical neurons with NMDA (50 uM) exposure for 3 hr assessed by relative neuronal death. In comparison to no drug treated group, LYS436 (p<0.0001), LYS739 (p<0.0001), LYS416 (p<0.01), biphalin (p<0.0001) and fentanyl (p<0.05) significantly decreased relative neuronal death. LYS739 (p<0.05) and LYS436 (p<0.05) showed better neuroprotection compared to biphalin. Compared to fentanyl alone, LYS436 (p<0.0001) and LYS739 (p<0.0001) displayed better neuroprotection in terms of LDH production. NTX reversed the effect of LYS436, LYS739, LYS416, biphalin and fentanyl.

FIG. 7 shows the effects of fentanyl analogs and biphalin on primary cortical neuronal ROS production upon exposure to 3 hr H/A and 24 hr reoxygenation. (‘*’ compared to no drug treated group; ‘#’ compared to biphalin treated group; *p<0.05, *p<0.01 **p<0.001, #p<0.05; data from 3 to 4 independent primary neuron isolations with 2-3 replicates treatment per isolation). All experimental groups were significantly different compared to normoxia (p<0.0001) and H₂O₂ (p<0.001). LYS436 (SEQ ID NO: 8) (p<0.001), LYS739 (SEQ ID NO: 10) (p<0.001), LYS416 (SEQ ID NO: 37) (p<0.01) and biphalin (p<0.05) significantly decreased ROS production compared to no drug treated group. LYS739 (p<0.05) showed better neuroprotection compared to biphalin in terms of ROS production. In comparison to fentanyl alone, LYS436 (p<0.001) and LYS739 (p<0.001) significantly reduced ROS production. NTX reversed the effect of biphalin, LYS436, LYS739 and LYS416.

FIGS. 8A, 8B, and 8C show the effects of fentanyl analog LYS739 (SEQ ID NO: 10) and biphalin (5 mg/kg, I.P. administration, 10 min after reperfusion), fentanyl (0.2 mg/kg, I.P. administration, 10 min after reperfusion) and non-selective OR antagonist NTX (1 mg/kg, I.P. administration, 10 min before surgery) or vehicle (0.9% saline) on edema and infarct formation in transient MCAO (60 min occlusion and 24 hr reperfusion). A) Representative TTC staining of brain slices from vehicle and drug treated mice. B) Brain edema ratio of brain in vehicle and drug treated groups. Fentanyl analog LYS739 (p<0.05) and biphalin (p<0.05) significantly decreased edema formation compared to vehicle treated group. In comparison to fentanyl alone, both LYS739 (p<0.05) and biphalin (p<0.05) significantly reduced edema formation. NTX reversed the effect of both biphalin (p<0.05) and LYS739 (p<0.05). NTX and FENT alone did not show any significant effect compared to vehicle treated group. C) Brain infarct ration in vehicle and drug treated mice. In comparison to vehicle treated group, fentanyl analog LYS739 (p<0.0001) and biphalin (p<0.0001) significantly reduced infarct formation in mice. Fentanyl and NTX alone did not show any improvement compared to saline treated group. Both biphalin (p<0.0001) and LYS739 (p<0.0001)) decreased infarct formation compared to fentanyl alone. NTX reversed the effect of biphalin (p<0.0001) and LYS739 (p<0.0001). (‘*’ compared to vehicle treated group; *p<0.05; ****p<0.0001; numbers indicated in the parenthesis in the figure columns denote to the number of experimental animals per group).

FIG. 9 shows the neurological score evaluation of mice 24 hr after ischemia and drug treatment. Both biphalin (p<0.05) and LSY739 (SEQ ID NO: 10) (p<0.05) improved neurological behavior compared to vehicle treated group whereas FENT and NTX alone did not improve any neurological score compared to vehicle treated group. NTX reversed the effect of biphalin (p<0.05) and fentanyl analog LYS739 (p<0.05). ‘*’ compared to vehicle treated group; *p<0.05; numbers indicated in the parenthesis in the figure columns denote to the number of experimental animals per group).

FIG. 10A-10E shows in vivo assays wherein bilateral RVM or intrathecal (i.th.) injections of LYS739 (SEQ ID NO: 10) (10 ug/0.5 uL) reversed tactile allodynia and thermal hyperalgesia in the Hargreaves test and the von Frey test, respectively, using neuropathic pain rats.

FIG. 11 shows stability of LYS739 in human plasma, e.g., HPLC profiles after incubation at 37° C. and peptide concentration (%) at various times.

FIG. 12 shows an example of the design of multifunctional opioid ligands with MOR/DOR agonist and KOR antagonist activity.

FIG. 13 shows an example of a scheme for the synthesis of multifunctional opioid analogs. (i) Boc-amino acid/BOP/HOBtNMM (1.1 eq/1.1 eq/1.1 eq/2 eq) in DMF for 3 hr at RT. (ii) 100% TFA for 20 min at 0° C. (iii) RP-HPLC: 10-50% of acetonitrile within 20 min. BOP: (Benzotriazol-1-yloxy)tris(dimethylamino) phosphoniumhexafluorophosphate; HOBt: 1-hydroxybenzotrazole; NMM: N-methyl morpholine. See FIG. 12 for Aaa, Bbb, Ccc, Ddd, and R.

FIG. 14 shows modifications of multifunctional ligands with MOR/DOR agonist and KOR antagonist activity.

FIG. 15 shows simple modifications of ligands for enhanced KOR activity.

FIG. 16 shows an example of a scheme for the synthesis of various multifunctional analogs. (i) Boc-amino acid/BOP/HOBNMM (1.1 eq/1.1 eq/1.1 eq/2 eq) in DMF for 2-4 h at RT. (ii) 100% TFA for 20 min at 0° C. (iii) RP-HPLC: 10-50% of acetonitrile within 20 min. (iv) Boc-cysteine/BOP/HOB/NMM (2.2 eq/2.2 eq/4 eq) in DMF for 3-5 h at RT (v) 5% TFA, 10 min, RT. (vi) DCC/HOBDIPEA. DCC: N,N′-dicyclohexylcarbodiimide; BOP: (Benzotriazol-1-yloxy)tris(dimethylamino) phosphoniumhexafluorophosphate; HOBt: 1-hydroxybenzotriazole; NMM: N-methyl morpholine. Mtt: 4-methyltrityl. M: 0, 1. See FIG. 14 for I, R, X, Fff, and Ggg.

### DESCRIPTION OF PREFERRED EMBODIMENTS

The present invention features multifunctional opioid receptor ligands (ORLs), acting as MOR agonists, DOR agonists, and KOR antagonists (or partial agonists). The present invention also features methods of use of said multifunctional ORLs, e.g., methods of treating pain or other conditions using peptides of the present invention.

As used herein, an “antagonist” refers to a type of receptor ligand or drug that blocks or dampens a biological response. An antagonist interferes in the natural operation of receptor proteins.

As used herein, a “partial agonist” refers to a type of receptor ligand or drug that binds to and activates a receptor, but is not able to elicit the maximum possible response that is produced by full agonists. Partial agonists may display some antagonistic behavior.

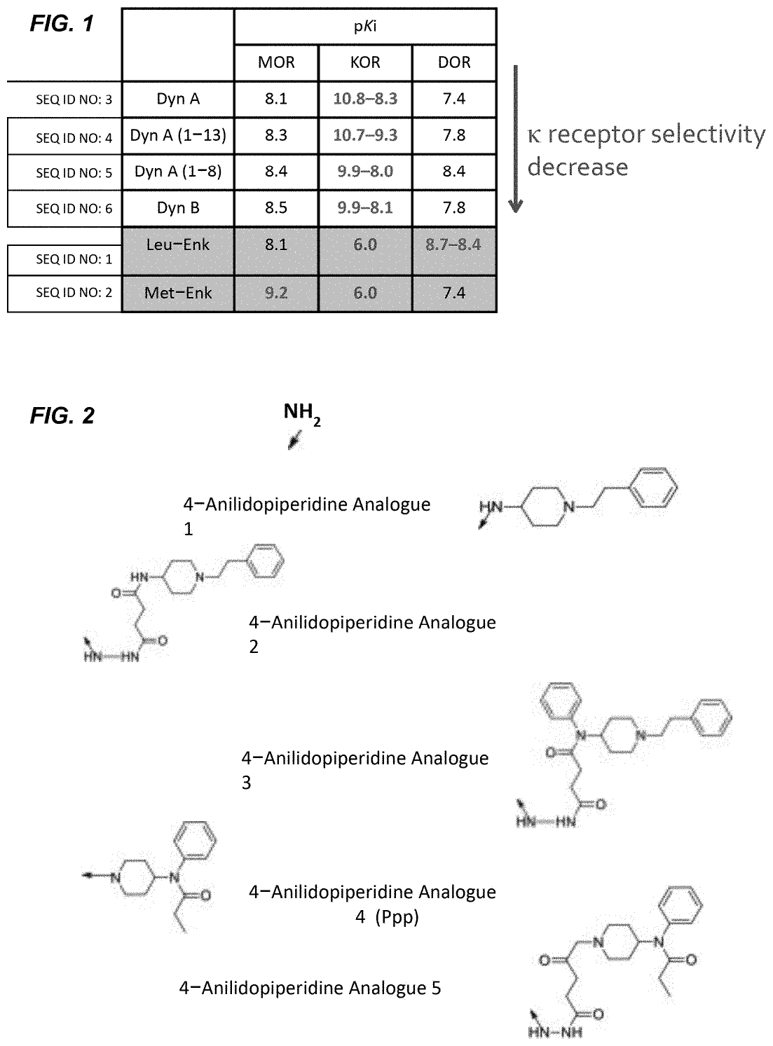

FIG. 1 shows the well-known structure-activity relationship (SAR) results of Dynorphin A (Dyn A) and enkephalins for opioid activities. Enkephalins shown are Leu-Enk (YGGFL, SEQ ID NO: 1) and Met-Enk (YGGFM, SEQ ID NO: 2). Dyn A is an endogenous kappa opioid receptor (KOR) ligand. The sequence for Dyn A is YGGFLRRIRPKLKWDNQ (SEQ ID NO: 3). (Note that the first five amino acids of Dyn A is Leu-Enk). Other peptides tested include a peptide containing the first 13 amino acids of Dyn A (Dyn A 1-13, YGGFLRRIRPKLK (SEQ ID NO: 4)), a peptide containing the first 8 amino acids of Dyn A (Dyn A 1-8, YGGFLRRI (SEQ ID NO: 5)), and Dyn B (YGGFLRRNFLVVT (SEQ ID NO: 6)). Without wishing to limit the present invention to any theory or mechanism, it appears that KOR selectivity decreases as the C-terminal residues of Dyn A are removed (e.g., Dyn A is more selective for KOR than is Leu-Enk). Without wishing to limit the present invention to any theory or mechanism, it is thought that residues following the first four amino acids of enkephalin, e.g., the residue(s) following the Phe/F residue of the enkephalin (or derivative) may be a region that helps make the ORL active for KOR, e.g., the residues following the first four amino acids of the enkephalin (or derivative thereof) may provide specificity for KOR.

The ORLs of the present invention comprise a peptide portion, e.g., a peptide analog derived from enkephalins (e.g., Leu-Enk (YGGFL, SEQ ID NO: 1) or Met-Enk (YGGFM, SEQ ID NO: 2)) and a tail portion linked to the C-terminus of the peptide portion. In some embodiments, the peptide portion comprises four residues (e.g., amino acids, analogs or derivatives thereof), occupying position 1, 2, 3, and 4. In some embodiments, the peptide portion comprises three residues (e.g., amino acids, analogs or derivatives thereof), occupying position 1, 2, and 4. The peptide portion may be based on the enkephalin sequence e.g., Leu-Enk (YGGFL, SEQ ID NO: 1) or Met-Enk (YGGFM, SEQ ID NO: 2).

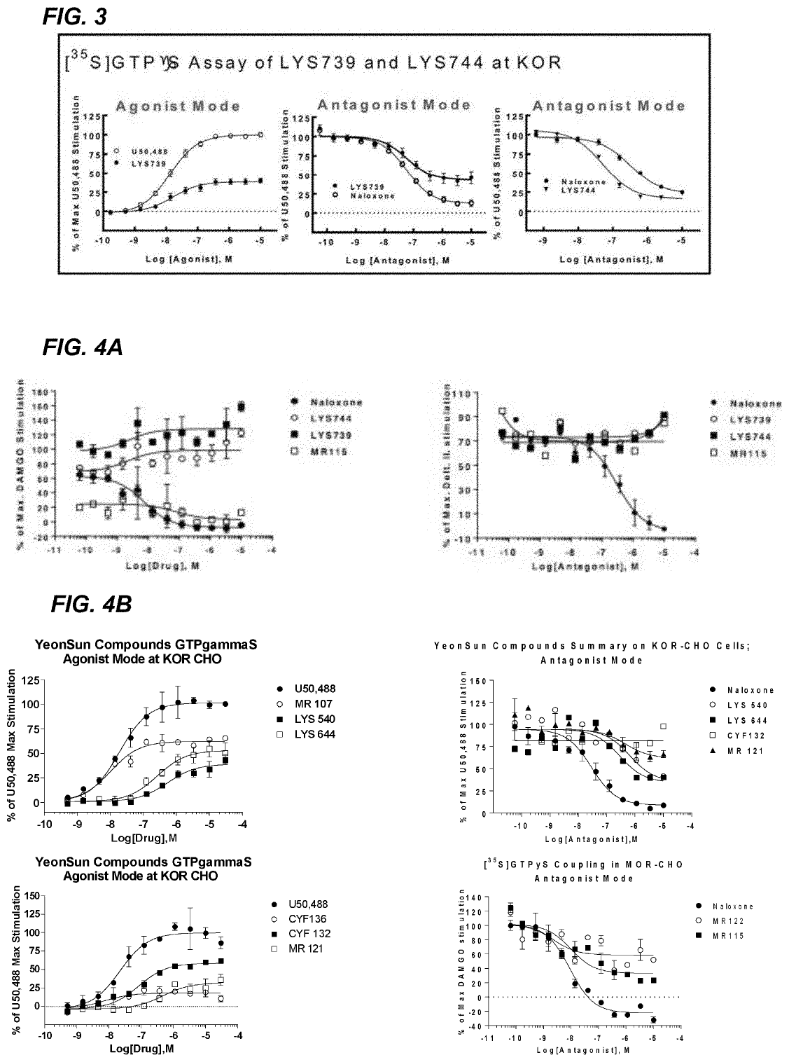

In some embodiments, the tail portion comprises a lipophilic molecule (e.g., a 4-anilidopiperidine moiety), e.g., the tail portion may comprise a residue or compound that increases the ipophiicity of the peptide portion. In some embodiments, the tail comprises a N-phenyl-N-piperidin-4-ypropionamide (Ppp) moiety. In some embodiments, the tail comprises —NH₂. Other non-limiting examples of tail portion molecules (tail compounds) are shown in FIG. 2. For reference, Lee et al. (Bioorganic and Medicinal Chemistry Letters 17, 2007, pp 2161-2165 & Journal of Medicinal Chemistry 50, 2007, pp 5528-5532) describes 4-anilidopiperidine analogues for biological activities at mu and delta opioid receptors.

Various non-limiting examples of formulas are presented herein for ORLs. For example, the present invention provides ORLs according to Formula 1 (Aaa-DBbb-Ccc-Ddd(X)-Eee). In some embodiments, Aaa is selected from Dmt and Tyr. In some embodiments, DBbb is selected from DAla, Nle, Pro, and DArg; In some embodiments, Ccc is selected from Gly, Phe(X), and Nal or is absent. In some embodiments, Ddd(X) is Gly, Phe(X), or Lys. Eee is a tail portion, wherein the tail portion is lipophilic. In some embodiments, X is Br. In some embodiments, X is selected from H, F, C, and Br. In some embodiments, Eee is selected from —NH₂ and a 4-anilidopiperidine moiety. In some embodiments, the 4-anilidopiperidine moiety comprises N-phenyl-N-piperidin-4-ylpropionamide (Ppp). The present invention is not limited to Formula 1. DXxx(X) refers to a D amino acid, and X refers to a halogen or other appropriate compound, e.g., H, C, F, or a methyl group. N-phenyl-N-piperidin-4-ylpropionamide may be abbreviated as Ppp. In some embodiments, residue 1 (e.g., Dmt, Aaa, etc.) comprises Dmt or Tyr. In some embodiments, residue 2 (DXxx, Bbb, etc.) comprises DAla, Nle, Pro, or DArg. In some embodiments, residue 3 (Gly, Ccc, etc.) comprises Gly, Phe, Phe(X), or Nal, wherein X may refer to H, Cl, F, methyl group, or any other appropriate modification of Phe. In some embodiments, residue 3 is absent. In some embodiments, residue 4 (Phe(X), Ddd, etc.) comprises Gly, Phe, Phe(X), wherein X may refer to H, C, F, methyl group, or any other appropriate modification of Phe. In some embodiments, the tail of the ORL comprises Ppp or NH₂. The present invention is not limited to the aforementioned formula molecules.

Table 1 below shows non-limiting examples of ORLs of the present invention. Note that the Phe residues in SEQ ID NO: 10, SEQ ID NO: 11, SEQ ID NO: 12, SEQ ID NO: 19, and SEQ ID NO: 20 are halogenated with F, and the Phe residue in SEQ ID NO: 15, SEQ ID NO: 16, and SEQ ID NOs: 26-30 are halogenated with Cl.

TABLE 1
Examples of ORLs

| | | Residue | | | |
|---|---|---|---|---|---|
| | | (Position from N-terminus | | | |
| | | to C-terminus) | | | |
| SEQ ID NO: | Compound | 1 | 2 | 3 | 4 |
| 7 | LYS729 | Tyr | DAla | Gly | Phe-NH₂ |
| 8 | LYS544 | Tyr | DAla | Gly | Phe-Ppp |
| 9 | LYS540 | Dmt | DAla | Gly | Phe-Ppp |
| 10 | LYS739 | Dmt | DNle | Gly | Phe(4-F)-Ppp |
| 11 | MR106 | Tyr | DNle | Gly | Phe(4-F)-NH₂ |
| 12 | MR107 | Dmt | DNle | Gly | Phe(4-F)-NH₂ |
| 13 | CYF132 | Dmt | DNle | Gly | Phe-NH₂ |
| 14 | LYS644 | Dmt | DNle | Gly | Phe-Ppp |
| 15 | LYS744 | Dmt | DNle | Gly | Phe(4-Cl)-Ppp |
| 16 | LYS702 | Dmt | DTic | — | Phe-Ppp |
| 17 | CYF136 | Dmt | DNle | Gly | Phe(4-Cl)-NH₂ |
| 18 | MR119 | Dmt | DNle | Gly | Phe(4-Br)-Ppp |
| 19 | MR111* | Dmt | DNle | Homocys | Phe(4-F)-Ppp |
| | (*see note | | | | |
| | below) | | | | |
| 20 | MR112* | Dmt | Homocys | Gly | Phe(4-F)-Ppp |
| | (*see note | | | | |
| | below) | | | | |
| 21 | MR124 | Dmt | DArg | Phe | Gly-Ppp |
| 22 | MR125 | Dmt | DArg | 1Nal | Gly-Ppp |
| 23 | MR110 | Dmt | DArg | Phe | Lys-Ppp |
| 24 | MR120 | Dmt | DArg | 1Nal | Lys-Ppp |
| 25 | MR122 | Dmt | DArg | — | 1Nal-Ppp |
| 26 | MR121 | Dmt | DArg | — | Phe(4-Cl)-Ppp |
| 27 | MR114 | Dmt | Pro | Trp | Phe(4-Cl)-Ppp |
| 28 | MR115 | Dmt | Pro | Phe | Phe(4-Cl)-Ppp |
| 29 | MR116 | Dmt | Pro | Phe(4-Cl) | Phe(4-Cl)-Ppp |
| 30 | MR123 | Dmt | Pro | Gly | Phe(4-Cl-Ppp |

Note
MR111 comprises two units of SEQ ID NO; 19, e.g., MR111 comprises (Dmt-DNle-Homocys-Phe(4-F)-Ppp)₂.
MR112 comprises two units of SEQ ID NO: 20, e.g., MR112 comprises (Dmt-Homocys-Gly-Phe(4-F)-Ppp)₂.

The ORLs of the present invention may be synthesized as appropriate (see, for example, Lee et al., Journal of Medicinal Chemistry 54, 2011, pp 382-886 & Lee, Current Protocols 98, 2019, e97). For example, the ORLs of the present invention may be synthesized by a protocol for liquid phase peptide synthesis (LPPS), e.g., using Boc-chemistry in high yields. In some embodiments halogen modification on the aromatic ring is on the para position, e.g., to help avoid unfavorable steric hindrance.

Table 2 shows analytical data of various multifunctional ORLs of the present invention. ^{a}FAB-MS (JEOL HX110 sector instrument) or MALDI-TOF. ^{b}Retention time on a Hewlett Packard 1100 [C-18, Vydac. 4.6 mm×250 mm, 5 μm, 10-100% of acetonitrile containing 0.1% TFA within 45 min 1 mL/min]. ^{c}http://www.vcclab.org/lab/alogps/. ^{d}Low resolution-Mass, n.d. not determined.

TABLE 2
Analytical data of ORLs

| ORL/ | | Molecular | HR MS^{a} (M-TFA + H)⁺ | | | |
|---|---|---|---|---|---|---|
| SEQ ID NO | Structure | Formula | observed | calculated | HPLC^{b} | ALOGPs^{c} |
| LYS729/7 | Tyr-DAla-Gly-Phe-NH₂ | C₂₃H₂₉N₅O₅ | 456.2239 | 456.2246 | 14.0 | 0.32 |
| LYS544/8 | Tyr-DAla-Gly-Phe-Ppp | C₃₇H₄₆N₆O₆ | 671.3579 | 671.3557 | 19.1 | 2.80 |
| LYS540/9 | Dmt-DAla-Gly-Phe-Ppp | C₃₉H₅₀N₆O₆ | 699.3852 | 699.3870 | 20.1 | 2.96 |
| LYS739/10 | Dmt-DNle-Gly-Phe(F)-Ppp | C₄₂H₅₅FN₆O₆ | 759.4247 | 759.4245 | 20.0 | 3.74 |
| MR106/11 | Tyr-DNle-Gly-Phe(4-F)-NH₂ | C₂₆H₃₄FN₅O₅ | n.d. | 515.2544 | 14.8 | 1.36 |
| MR107/12 | Dmt-DNle-Gly-Phe(4-F)-NH₂ | C₂₈H₃₈FN₅O₅ | n.d. | 543.2857 | 15.7 | 1.75 |
| CYF132/13 | Dmt-DNle-Gly-Phe-NH₂ | C₂₈H₃₉N₅O₅ | 526.3035 | 526.3030 | 16.5 | 1.69 |
| LYS644/14 | Dmt-DNle-Gly-Phe-Ppp | C₄₂H₅₆N₆O₆ | 741.4325 | 741.4340 | 19.3 | 3.66 |
| LYS744/15 | Dmt-DNle-Gly-Phe(4-Cl)-Ppp | C₄₂H₅₅CIN₆O₈ | 775.3995 | 774.3950 | 21.8 | 4.18 |
| LYS702/16 | Dmt-DTic-Phe-Ppp | C₄₄H₅₀CIN₅O₅ | 764.3632 | 765.3658 | 23.0 | 4.91 |
| CYF136/17 | Dmt-DNle-Gly-Phe(4-Cl)-NH₂ | C₂₈H₃₈CIN₅O₅ | 560.2653 | 560.2640 | 16.0 | 2.36 |
| MR111/19 | (Dmt-DNle-Homocys- | C₈₈H₁₁₆F₂N₁₂O₁₂S₂ | 1635.5^{d} | 1635.8324 | 25.4 | 5.58 |
| | Phe(4-F)-Ppp)₂ | | | | | |
| MR112/20 | (Dmt-Homocys-Gly-Phe(4-F)- | C₈₀H₁₀₀F₂N₁₂O₁₅S₂ | 1524.4^{d} | 1523.7072 | 23.4 | 4.89 |
| | Ppp)₂ | | | | | |
| MR121/26 | Dmt-DArg-Phe(4-Cl)-Ppp | C₄₀H₅₃CIN₈O₅ | 761.3900 | 761.3906 | 20.3 | 3.00 |
| MR114/27 | Dmt-Pro-Trp-Phe(4-Cl)-Ppp | C₅₀H₅₈CIN₇O₆ | 888.4208 | 868.4216 | 26.0 | 5.20 |
| MR116/29 | Dmt-Pro-Phe(4-Cl)-Phe(4-Cl)- | C₄₈H₅₆CIN₆O₈ | 883.3693 | 883.3717 | 23.9 | 5.23 |
| | Ppp | | | | | |

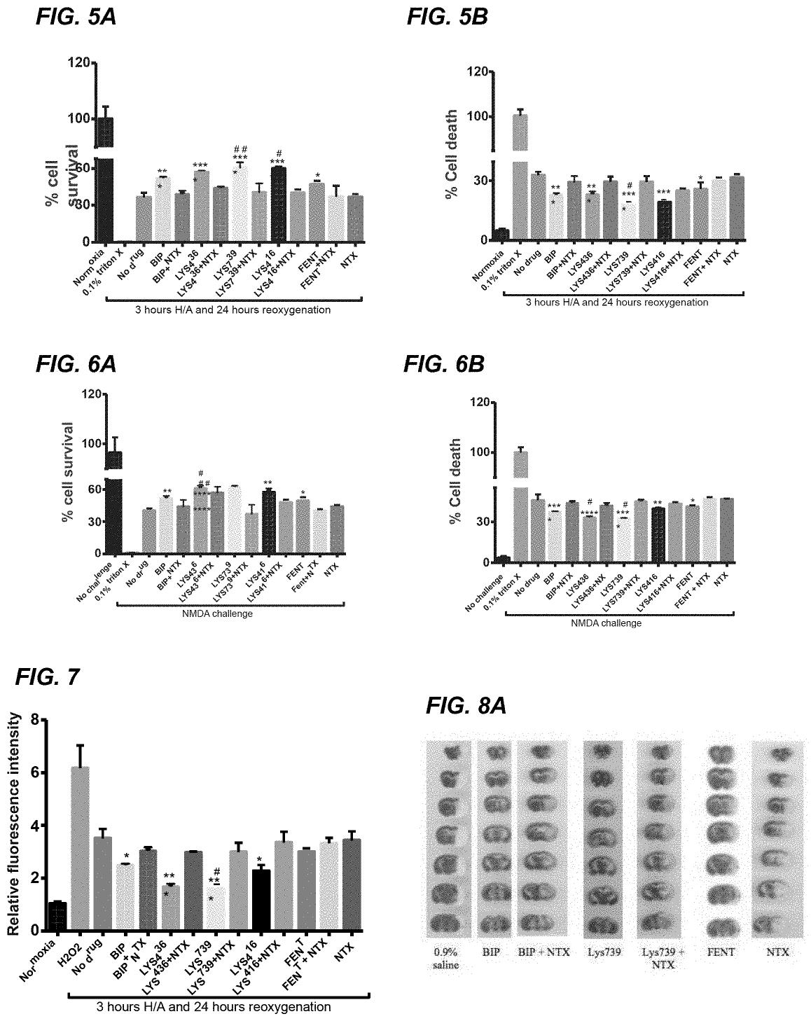

As shown in Table 3. Table 41, Table 4.2, and FIG. 3 it was surprisingly discovered that the ORL LYS739 (SEQ ID NO: 10 Dmt-DNle-Gly-Phe(4-F)-Ppp) interacts with KOR (Kᵢ=0.89 nM) as well as MOR (Kᵢ=0.02 nM) and DOR (Kᵢ=0.40 nM). Considering well-known structure-activity relationships (SAR) of enkephalin analogues, the sub nanomolar range of binding affinity of LYS739 (SEQ ID NO: 10) at the KOR was unexpected and could not be predicted. LYS739 turned out to be the first potent MOR/DOR agonist (IC₅₀: 0.26 nM, and 0.37 nM in GPI, and MVD, respectively) and KOR partial agonist/antagonist among ORLs. In GTP-γ-assay. LYS739 showed mixed partial agonist (EC₅₀=21 nM, Eₘₐₓ=39%)/antagonist activity (EC₅₀=60 nM, Eₘₐₓ=65%) for KOR. Note that in Table 4.1 and Table 4.2, potency and efficacy reported as mean±SEM from each experiment (n=3) independent experiments for both modes; curves use the mean value of each point from each experiment combined together; n.d. is not determined. It was surprisingly discovered that the ORL LYS744 (SEQ ID NO: 15) interacts with KOR, as well as MOR and DOR. Interestingly, LYS744, containing a Phe(4-Cl) residue instead of a Phe(4-F), showed full antagonist activity (IC₅₀=52 nM, Iₘₐₓ=122%) in the assay.

TABLE 3
Binding affinities of ORLs at MOR, DOR, and KOR

| | | Ki (nM) | | |
|---|---|---|---|---|
| | | MOR | DOR | KOR |
| SEQ ID NO: | Compound | [³H]DAMGO | [³H]DPDPE | [³H]U69, 593 |
| 7 | LYS729 | 2.8 | 300 | 230 |
| 8 | LYS544 | 26 | 5.2 | 190 |
| 9 | LYS540 | 0.38 | 0.36 | 21 |
| 10 | LYS739 | 0.02 | 0.4 | 0.89 |
| 11 | MR106 | 2.3 | 9.3 | 30 |
| 12 | MR107 | 0.15 | 0.64 | 0.98 |
| 13 | CYF132 | n.d | n.a | 3.4 |
| 14 | LYS644 | 0.39 | 0.18 | 4.8 |
| 15 | LYS744 | 0.08 | 0.10 | 1.3 |
| 16 | LYS702 | 0.45 | 0.76 | n.d |

TABLE 4.1
Functional Activities of LYS739 (SEQ
ID NO: 10) at MOR, DOR, and KOR

| [³⁵S]GTP-γ-S binding assay | | | | | | |
|---|---|---|---|---|---|---|
| | | | Antagonist | | | |
| | | | IC₅₀ | | | |
| Agonist | | | (nM) | IC₅₀ (nM) | | |
| EC₅₀ (nM) (Eₘₐₓ %) | | | (Iₘₐₓ %) | GPI | MVD | GPI |
| hDOR | rMOR | hKOR | hKOR | (μ) | (δ) | (k) |
| 0.07 (48^{a}) | 0.29 (98^{a}) | 21 (39^{b}) | 60 (65)^{c} | 0.26 | 0.37 | n.d. |

^{a}[total bound-basal]/[basal-nonspecific] × 100.
^{b}Relative % of 10 μm U50, 488 stimulation.
^{c}Relative % of naloxone blocking 100 nM U50, 488 stimulation.
n/d: not determined.

TABLE 4.2
Functional Activities of LYS744 (SEQ
ID NO: 15) at MOR, DOR, and KOR

| [³⁵S]GTP-γ-S binding assay | | | | | | |
|---|---|---|---|---|---|---|
| | | | Antagonist | | | |
| | | | IC₅₀ | | | |
| Agonist | | | (nM) | IC₅₀ (nM) | | |
| EC₅₀ (nM) (Eₘₐₓ %) | | | (Iₘₐₓ %) | GPI | MVD | GPI |
| hDOR | rMOR | hKOR | hKOR | (μ) | (δ) | (k) |
| 0.07 (37^{a}) | 0.14 (58^{a}) | <10 at 10 μm^{b} | 52 (122)^{c} | 1.3 | 1.9 | n.d. |

^{a}[total bound-basal]/[basal-nonspecific] × 100.
^{b}Relative % of 10 μm U50, 488 stimulation.
^{c}Relative % of naloxone blocking 100 nM U50, 488 stimulation.
n/d: not determined.

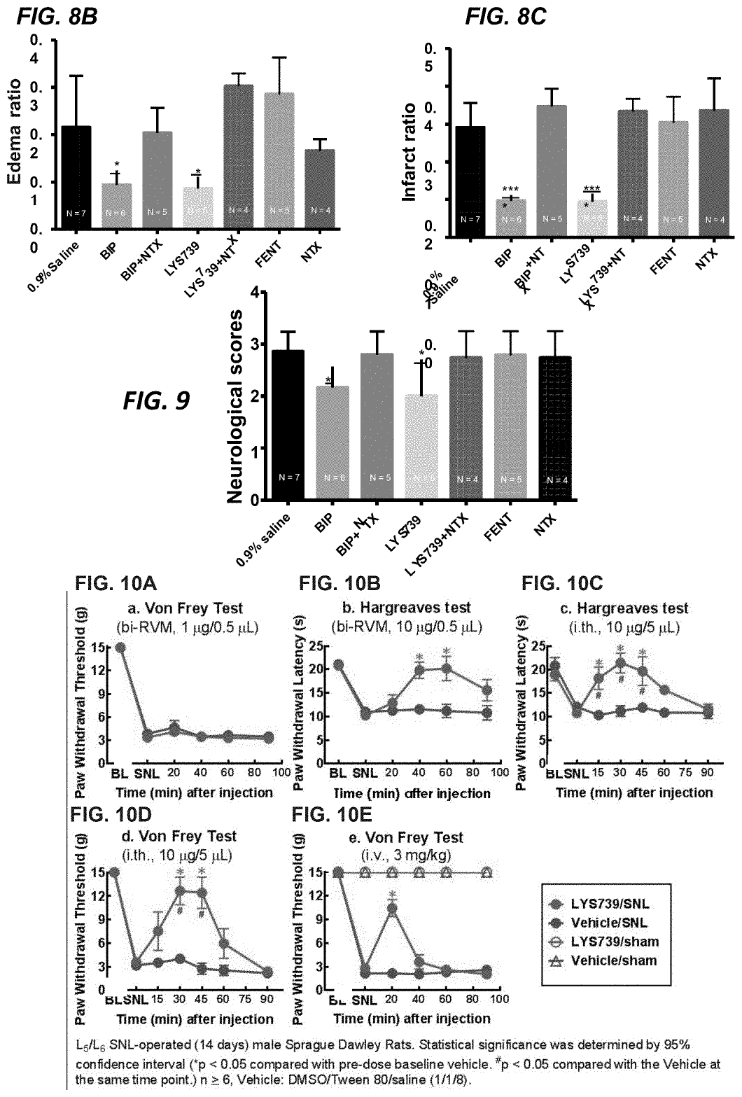

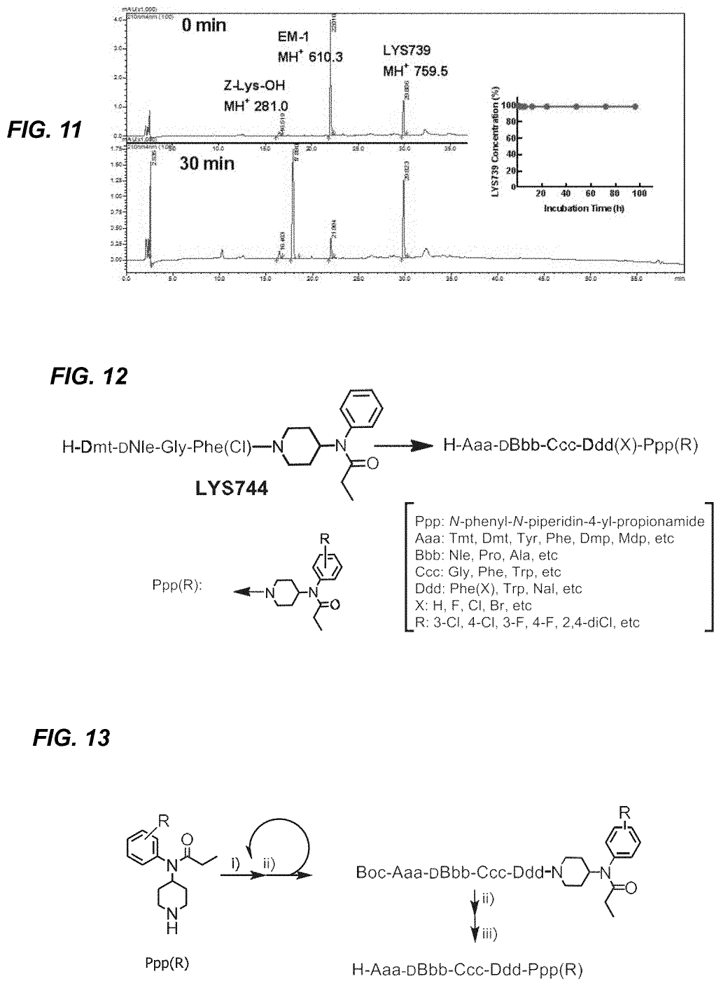

Preliminary in vivo studies of LYS739 (SEQ ID NO. 10) showed that intrathecal (i.th.) administration of LYS739 (SEQ ID NO: 10) at 10 μg/5 μl can reverse thermal hyperalgesia and reverse tactile allodynia in L₅/L₆ SNL-operated male SD rats. For example, FIG. 4A shows [³⁵S]GTPγS assays: MOR (left) and DOR (right) antagonist modes. LYS739 (SEQ ID NO: 10), LYS744 (SEQ ID NO: 15), and MR115 (SEQ ID NO: 28) do not possess antagonist activity at MOR and DOR. FIG. 4B shows [³⁵S]GTPγS assays: KOR agonist (left column) and antagonist (right column) modes. LYS540 (SEQ ID NO: 9), LYS644 (SEQ ID NO: 14), and MR121 (SEQ ID NO. 126) are partial agonist/antagonist at KOR. CYF132 (SEQ ID NO: 13), and MR107 (SEQ ID NO. 12) are observed as a partial agonist at KOR. In FIG. 10A, statistical significance was determined by 95% confidence interval (\hat{ }P<0.05 compared with pre-dose SNL baseline vehicle; #p<0.05 compared with the vehicle at the same time point; n>6). Vehicle was DMSO/Tween 80/Saline (1:1:8). Intravenous (i.v.) administration of LYS739 (SEQ ID NO: 10) (3 mg/Kg) in L₅/L₆ SNL-operated male SD rats showed reversal of thermal hyperalgesia. This represents high potency of analgesic effects through MOR (and DOR).

For reference, Table 5 lists examples of ORLs with various tail portions (e.g., NH₂ and Tail Compounds 1-5). Structures of the Tails (e.g., anilidopiperidine moieties) can be found in FIG. 2. Table 5 also shows lipophilicity values and MOR/DOR agonist activities of the ORLs. Note that SEQ ID NO: 8 refers to both LYS544 and LYS436.

TABLE 5
MOR/DOR agonist activities of C-terminal
modified lipophilic enkephalin analogues

| ORL and | | | Kᵢ (nM) |
|---|---|---|---|
| Structure | Tail | aLogP | MOR/DOR |
| LYS729 (SEQ ID NO: 7) | -NH₂ | 0.32 | 2.8/300 |
| Tyr-DAla-Gly-Phe-NH₂ | | | |
| LYS416 (SEQ ID NO: 31) | 4-Anilidopiperidine | 2.93 | 14/14 |
| Tyr-DAla-Gly-Phe-Tail | analogue 1 | | |
| LYS620 (SEQ ID NO: 32) | 4-Anilidopiperidine | 2.63 | 1.2/3.7 |
| Tyr-DAla-Gly-Phe-Tail | analogue 2 | | |
| LYS429 (SEQ ID NO: 33) | 4-Anilidopiperidine | 4.04 | 1.1/6.1 |
| Tyr-DAla-Gly-Phe-Tail | analogue 3 | | |
| LYS544 (or LYS436) | 4-Anilidopiperidine | 2.80 | 23/0.69 |
| (SEQ ID NO: 8) | analogue 4 (Ppp) | | |
| Tyr-DAla-Gly-Phe-Ppp | | | |
| LYS437 (SEQ ID NO: 34) | 4-Anilidopiperidine | 2.13 | 5.7/3.2 |
| Tyr-DAla-Gly-Phe-Tail | analogue 5 | | |

The present invention also features ORLs that are derived from LYS739 (SEQ ID NO: 10), e.g.. LYS739 analogs. In some embodiments, the ORLs are obtained by modifying LYS739 (SEQ ID NO: 10) by substitution, dimerization, and/or cyclization. Modifications may involve the incorporation of an unnatural amino acid and/or constrained amino acids. For example, in some embodiments, Dmt is substituted with Tml. In some embodiments, the ORL comprises Mdp.

In some embodiments, the ORL comprises a bivalent ligand in some embodiments, a disulfide bond is used to link two monomeric pharmacophores. For example, a disulfide bond may be used through a homocysteine residue at position 2 (or 3). In some embodiments. ORLs comprise cyclic structures, e.g., the ORLs are cyclic and retain the pharmacophoric structure for the receptors within a constrained structure, e.g., since linear peptide ligands can be flexible even with multiple modifications due to high flexibility of enkephalins. Cyclization may be through the formation of various bonds such as a disulfide and a lactam but is not limited to these mechanisms.

In some embodiments, the ORLs are bifunctional ligands. In some embodiments, the ORLs are trifunctional ligands. In some embodiments. ORLs are constructed based on an enkephalin tetrapeptide (Tyr-Gly-Gly-Phe-NH2. SEQ ID NO: 46). In some embodiments, ORLs are constructed using EM-1 and/or DALDA. The present invention features ORL designs using EM-1 (Tyr-Pro-Trp-Phe-NH₂, SEQ ID NO: 35) and DALDA (Tyr-DArg-Phe-Lys-NH₂, SEQ ID NO: 36). The present invention also features ORLs using EM-2 (Tyr-Pro-Phe-Phe-NH, SEQ ID NO: 45).

Various ORLs (e.g., analogs of LYS739 (SEQ ID NO: 10)) were tested for their binding affinities at MOR, DOR, and KOR using [³H]DAMGO, [³H]DPDPE, and [³H]U69,593, respectively, in the membranes from stable HEK cells constitutively expressed the respective opioid receptors. Analogues with particular binding affinity (Ki<10 nM for MOR and DOR; Ki<30 nM for KOR) as well as others were tested for receptor functional activity in the [³⁵S]-GTPγS assay. In this assay, antagonist activity at all three receptors expressed in CHO cells were determined by the inhibition of stimulation caused by 100 nM of control agonist (DAMGO for MOR, SNC80 for DOR, U50,488 for KOR) in a 96-well plate. Table 6 summarized in vitro biological activities of multifunctional ligands at MOR. DOR, and KOR with a Ppp group at the C-terminus. (Note. ^{a}=Competition analyses were carried out using membrane preparations from transfected HEK cells that constitutively expressed the respective receptor types; ^{b}=[³H]DAMGO, K_{d}=0.85 nM; ^{c}=[³H]DPDPE, Kd=0.50 nM; ^{d}=[³H]U69,593, Kd=5.3 nM; ^{e}=Expressed in CHO cells; ^{f}=Mean±SEM of the % relative to 10 μM U50,488 stimulation; ^{g}=Mean±SEM of the % relative to 10 μM naloxone inhibition of 100 nM U50,488; ^{h}=at 10 μM; ⁱ=cAMP test.

TABLE 6
Biological data of various ORLs

| SEQ | | | | | KOR^{e} | | | | |
|---|---|---|---|---|---|---|---|---|---|
| ID | | Kₗ, nM^{a} | | | [³⁵S]GTPγS-binding assay | | | | KOR |
| NO: | ligand | MOR^{b} | DOR^{c} | KOR^{d} | EC₅₀, nM | Eₘₐₓ, %^{f} | IC₅₀, nM | Iₘₐₓ, %^{g} | function |
| 7 | LYS729 | 2.8 | 300 | 230 | — | <30^{h} | — | <10^{h} | no function |
| 8 | LYS544 | 26 | 5.2 | 190 | — | <10^{h} | — | <10^{h} | no function |
| 9 | LYS540 | 0.38 | 0.36 | 21 | 540 | 40 | 630 | 49 | weak |
| 10 | LYS739 | 0.02 | 0.40 | 0.89 | 21 | 39 | 60 | 65 | partial agonist/ |
| | | | | | | | | | antagonist |
| 11 | MR106 | 2.3 | 9.3 | 30 | — | 70^{h} | | | weak |
| 12 | MR107 | 0.15 | 0.64 | 0.98 | 10 | 62 | 250 | 37 | partial agonist |
| 13 | CYF132 | 0.50 | 1.2 | 5.0 | 84 | 59 | n.c.* | — | partial agonist |
| 14 | LYS644 | 0.39 | 0.18 | 4.8 | 260 | 53 | 290* | 70 | weak |
| 15 | LYS744 | 0.08 | 0.10 | 1.3 | — | <10^{h} | 52 | 122 | antagonist |
| 16 | LYS702 | 0.45 | 0.76 | 890 | — | <10^{h} | | | no function |
| 17 | CYF136 | 2.9 | 0.61 | 156 | 7.0 | 18 | 66 | 60 | partial |
| | | | | | | | | | agonist/ |
| | | | | | | | | | antagonist |
| 19 | MR111 | 1.3 | 1.8 | 8.1 | 21 | <20 | 110 | 80 | antagonist |
| 20 | MR112 | 23 | 11 | 67 | — | <10^{l} | — | <10^{l} | No function |
| 26 | MR121 | 7.8 | 140 | 2.1 | 470 | 32 | 450* | 38 | weak |
| 27 | MR114 | 9.6 | 16 | 15 | — | <10^{l} | 39^{l} | >100^{l} | antagonist |
| 29 | MR116 | 37 | 49 | 21 | >1,000 | — | — | — | weak |

Analogues were tested for their activity at KOR, and GTPγS assays were performed at the MOR and DOR for LYS739 (SEQ ID NO: 10) and LYS744 (SEQ ID NO: 15) (see FIG. 4A). The assay results show that two ligands are pure agonists for the MOR and DOR. LYS744 (SEQ ID NO: 15). MR111 (SEQ ID NO: 19) and MR114 (SEQ ID NO:27) are pure antagonists for the KOR, which has a potential to reduce KOR related side effects. LYS540 (SEQ ID NO 9). LYS644 (SEQ ID NO:14), and MR121 (SEQ ID NO:26) are weak partial agonist/antagonist (see FIG. 5B). CYF136 (SEQ ID NO: 17) also revealed partial agonist/antagonist activity.

The present invention also features ORLs having half-lives longer than 4 hours. For example, in some embodiments the ORL has a half-life longer than 1 hour. In some embodiments, the ORL has a half-life longer than 2 hours. In some embodiments, the ORL has a half-life longer than 3 hours. In some embodiments, the ORL has a half-life longer than 4 hours. In some embodiments, the ORL has a half-life longer than 5 hours. In some embodiments, the ORL has a half-life longer than 10 hours. In some embodiments, the ORL has a half-life longer greater than 24 hours.

In some embodiments, the ORL is 4 amino acids in length. In some embodiments, the ORL is 5 amino acids in length. In some embodiments, the ORL is 6 amino acids in length. In some embodiments, the ORL is 7 amino acids in length. In some embodiments, the ORL is 8 amino acids in length. In some embodiments, the ORL is 9 amino acids in length. In some embodiments, the ORL is 10 amino acids in length. In some embodiments, the ORL is more than 10 amino acids in length.

In some embodiments, the ORL is between 4 to 6 amino acids in length. In some embodiments, the ORL is between 4 to 7 amino acids in length. In some embodiments, the ORL is between 4 to 8 amino acids in length. In some embodiments, the ORL is between 4 to 9 amino acids in length. In some embodiments, the ORL is between 4 to 10 amino acids in length. In some embodiments, the ORL is between 4 to 20 amino acids in length. In some embodiments, the ORL is between 4 to 30 amino acids in length. In some embodiments, the ORL is between 4 to 40 amino acids in length. In some embodiments, the ORL is between 4 to 50 amino acids in length.

As shown in FIG. 10A-10E, bilateral RVM or intrathecal (i.th.) injections of LYS739 (10 μg/0.5 μL) significantly reversed tactile allodynia and thermal hyperalgesia in the Hargreaves test and the von Frey test, respectively, using L₅/L₆ SNL-operated male Sprague Dawley (SD) rats. A relatively low dose of intravenous (i.v.) (3 mg/kg) LYS739 also significantly attenuated nerve injury induced tactile allodynia in the rats. The peak time of antiallodynic effect of LYS739 was observed 20 min post-administration, with a mean paw withdrawal threshold significantly higher than that of vehicle-treated injury. LYS739 is considered to possess great potential in having both potent and efficacious analgesia after systemic administration and is capable of crossing the BBB (comparing i.t. with i.v. administration). Efforts to improve the biological activity of enkephalin also increased metabolic stability due to the three non-natural amino acid modifications. LYS739 was very stable in human plasma. No degradation was observed after 96 h incubation at 37° C., while EM-1, which was used to validate the plasma's activity as a reference compound, was degraded very quickly in an hour (FIG. 11).

FIG. 12 shows an example of the design of multifunctional opioid ligands with MOR/DOR agonist and KOR antagonist activity. As an example, the Ppp(R) group may be retained at the C-terminus for these modifications. In some embodiments, Tyr residue may be replaced with a Dmt residue or a Tmt residue, which is more sterically hindered due to an extra methyl group. In some embodiments, Mdp, which has been known to reverse KOR activity dramatically from agonist to antagonist, may be used to investigate the reversal or enhancement of KOR activity and selectivity. This modification is also known to delete MOR/DOR agonist activity, and therefore may result in the discovery of a highly selective enkephalin analogue with antagonist activity at KOR. The Phe residue in ligands may be substituted with Phe(p-X) for altering receptor selectivity and inducing KOR interaction. The modifications at position 4 with a halogen may create novel analogues showing diverse KOR activities. Halogen modification on the aromatic ring may be limited to the para position, e.g., due to unfavorable steric hindrance effects that can lead to suboptimal binding interactions.

FIG. 13 shows a scheme for the synthesis of multifunctional opioid analogs. For example, the present invention may feature a protocol for liquid phase peptide synthesis (LPPS) using Bo-chemistry, which allows synthesis of peptides on the multi gram-scale through a robust procedure. This method may benefit from unnecessary C-terminal masking and unmasking steps due to a PPP(R) group at the C-terminus, and unnecessary side group protection. Commercially available PPP(R) may be used for the synthesis. During synthesis, intermediate peptides may be easily isolated by a simple precipitation using diethyl ether, which may avoid tedious purification steps. The simple isolation of intermediate peptides may allow short synthetic steps with high purity (ex. ≥98% for LYS744) in good overall yields (ex. ≥40% for LYS744). Compounds synthesized may be characterized by RP-HPLC (Hewlett Packard 1100, C-18, Vydac, 4.6 mm×250 mm, 5 μm, 10-90% of acetonitrile containing 0.1% TFA within 40 min, 1 mL/min.), HR-MS (Brucker 9.4 T Apex-Qh FTICR, JEOL HX110 sector instrument, or Brucker Ultraflex III MALD TOF-TOF), and NMR (Brucker DRX-600).

In some embodiments, competitive radioligand binding assays and cell based functional assays are performed. In some embodiments, compounds with a binding affinity of about Kᵢ<100 nM for MOR, DOR and KOR may be tested for receptor functional activity in a cyclic AMP assay. Compounds that show partial agonist (EC₅₀<100 nM, Eₘₐₓ<40%) or antagonist activity (IC₅₀<100 nM, Iₘₐₓ>60%) at the KOR and agonist activity (EC₅₀<100 nM, Eₘₐₓ>70%) at the MOR and DOR in the cyclic AMP assay may be used for off-target screening. In some embodiments, binding affinity (Kᵢ) can be determined by radioligand competition analysis using [³H]Diprenorphine for MOR, DOR, and KOR, in cell membrane preparations from stably transfected CHO cells expressing respective receptor types.

In some embodiments, cAMP accumulation may be measured. As a non-limiting example, in some embodiments, MOR, DOR, and KOR-CHO cells as above may be plated in 96 well culture microplates, and recovered overnight. The cells may then be serum starved for 20 minutes in serum free medium with 500 μM IBMX, followed by 15 minutes of treatment with 500 μM IBMX, 100 μM forskolin, and concentration curves of experimental drug or reference agonist (DAMGO for MOR, SNC80 for DOR, U50,488 for KOR). Antagonist measurements may be performed using a concentration curve of experimental drug or reference antagonist combined with a fixed concentration of agonist (EC₉₀). The incubation may be terminated, and lysates may be combined with ˜1 pmol of [H]cAMP and 7 μg of recombinant PKA, and incubated for 1 hour at room temperature. The reaction may be harvested and analyzed to generate potency (EC₅₀/IC₅₀) and efficacy (Eₘₐₓ/Iₘₐₓ) values for each compound. In some embodiments, off-target activities of compounds selected from in vitro analysis may be confirmed by the screening offered by the National Institute of Mental Health's Psychoactive Drug Screening Program (contract #HHSN-271-2008-025C (51). Note LYS739 did not show any off-target activities. In some embodiments, compounds with binding affinity below 100-fold vs. MOR/DOR/KOR for the other off-target receptors may be excluded from further studies.

In some embodiments, NMR analysis and/or computer modeling experiments are used to help identify structural features of enkephalin that may be important for KOR antagonist activity.

FIG. 14 shows a non-limiting example of design of multifunctional ligands. The multifunctional ligands may be synthesized with high efficacy and high potential bioavailability. Modifications may include i) cyclization, ii) dimerization, and iii) C-terminal elongation. In some embodiments, cyclic ligands that retain the pharmacophore structure for the receptors within a constrained ring structure may be synthesized (in some embodiments, cyclic peptides may possess high potential to increase both biological activity and bioavailability due to their conformational rigidity).

Cyclization may be achieved through the formation of various bonds such as a disulfide and a lactam bond. Bivalent ligands may be built on the pharmacophore structure. In order to link two monomeric pharmacophores, a disulfide bond may be utilized, e.g., through a homocysteine residue at position 2 (or 3). In some embodiments, the C-terminal chain elongation may be applied to enhance the KOR activity. For example, this modification may feature attachment of Leu⁵, Arg⁶, Ile⁸, and Arg⁹ residues in the dynorphin structure to a tetrapeptide scaffold.

The present invention also provides modifications of several known opioid ligands, such as endomorphin-1 (EM-1) (Kᵢ+=0.36 nM for MOR with 4,000- and 15,000-fold preference over DOR and KOR, respectively) and [DArg², Lys⁴]-dermorphin (DALDA) (Ki=1.69 nM for MOR with 11,000- and 2,500-fold preference over DOR and KOR, respectively) (see FIG. 15).

For example, in some embodiments, the ORL is derived from DALDA, e.g., according to Formula 5: Aaa-DArg-Ccc-Ddd-Eee. In some embodiments, Aaa is selected from Tyr or Dmt; Ccc is selected from Phe, Phe(X), or 1Nal; Ddd is selected from Lys, Gly or is absent; Eee is a 4-anilidopiperidine moiety (e.g., Ppp); and X is selected from F, Cl, or Br. In some embodiments, Ppp comprises Ppp(R), wherein R comprises a hydrogen or a halogen. In some embodiments, R is selected from 3-Cl, 4-Cl, 3-F, 4-F, and 2,4-diCl. In some embodiments, the ORL is SEQ ID NO: 21, SEQ ID NO: 22, SEQ ID NO: 23, SEQ ID NO; 24, SEQ ID NO: 25, SEQ ID NO: 26, SEQ ID NO: 42, or SEQ ID NO: 43.

In some embodiments, the ORL is derived from EM-1 or EM-2, e.g., the ORL is according to Formula 6: Aaa-Pro-Ccc-Phe(X)-Eee. In some embodiments, Aaa is selected from Tyr or Dmt; Ccc is selected from Trp, Phe, Gly, or Phe(X); Eee is a 4-anilidopiperidine moiety (e.g., Ppp), and X is selected from F, Cl, or Br. In some embodiments, Ppp comprises Ppp(R), wherein R comprises a hydrogen or a halogen. In some embodiments, R is selected from 3-Cl, 4-Cl, 3-F, 4-F, and 2,4-diCl. In some embodiments, the ORL is selected from SEQ ID NO: 27, SEQ ID NO: 28, SEQ ID NO: 29, or SEQ ID NO: 30.

In some embodiments, the ORL is derived from FE20066, e.g., according to Formula 7: DPhe-DPhe-DNle-Ddd-Eee. In some embodiments, Ddd is selected from DArg or DLys, and Eee is a 4-anilidopiperidine moiety (e.g., Ppp). For example, in some embodiments, Ddd is DArg and Eee is Ppp. In some embodiments. Ddd is DLys and Eee is Ppp. In some embodiments, Ppp comprises Ppp(R), wherein R comprises a hydrogen or a halogen. For example, in some embodiments, Ddd is DArg and Eee is Ppp(R), wherein R comprises a hydrogen or a halogen (e.g., Cl, F, Br). In some embodiments, Ddd is DLys and Eee is Ppp(R), wherein R comprises a hydrogen or a halogen (e.g., Cl, F, Br). In some embodiments, R is selected from 3-Cl, 4-Cl, 3-F, 4-F, and 2,4-diCl. In some embodiments, the ORL is according to SEQ ID NO: 44.

The present invention also provides an opioid receptor ligand dimer according to SEQ ID NO: 19. The present invention also provides an opioid receptor ligand dimer according to SEQ ID NO: 20.

FIG. 16 shows a non-limiting example of synthesis of compounds. In some embodiments, synthesis features LPPS using standard Boo-chemistry. For the synthesis of cyclic compounds, highly acid labile protecting groups may be used. In some embodiments, after chain elongation, linear compounds may be cyclized in a highly diluted solution to avoid intermolecular dimerization.

In some embodiments, modifications to the peptide ligand (e.g., incorporation of a Ppp group at the C-terminus) enhances metabolic stability and/or lipophilicity and/or blood brain barrier (BBB)/central nervous system (CNS) permeability.

Table 7 shows analytical data for several multifunctional opioid receptor ligands (ORLs) with a Ppp group at the C-terminus. For reference: ^{a}FAB-MS (JEOL HX110 sector instrument) or MALDI-TOF. ^{b}Retention time on a Hewlett Packard 1100 [C-18, Vydac, 46 mm×250 mm, 5 μm, 10-100% of acetonitrile containing 0.1% TFA within 45 min. 1 mL/min]. ^{c}http://www.vcclab.org/lab/alogps/

TABLE 7
Analytical data of ORLs

| ORL/ | | Molecular | HR MS^{a} (M-TFA + H)⁺ | | HPLC^{b} | |
|---|---|---|---|---|---|---|
| SEQ ID NO | Structure | Formula | observed | calculated | % ACN | ALOGPs^{c} |
| MR119/18 | Dmt-DNle-Gly-Phe(4-Br)-Ppp | C₄₂H₅₅BrN₆O₆ | 821.3415 | 819.3445 | 25.4 | 4.25 |
| MR124/21 | Dmt-DArg-Phe-Gly-Ppp | C₄₂H₅₇N₉O₆ | 784.4506 | 784.4511 | 16.6 | 1.92 |
| MR125/22 | Dmt-DArg-1Nal-Gly-Ppp | C₄₆H₅₉N₉O₅ | 834.4661 | 834.4667 | 19.9 | 2.83 |
| MR110/23 | Dmt-DArg-Phe-Lys-Ppp | C₄₆H₅₈N₁₀O₅ | 855.5238 | 855.5246 | 13.9 | 1.89 |
| MR120/24 | Dmt-DArg-1Nal-Lys-Ppp | C₅₀H₅₈N₁₀O₆ | 905.5389 | 905.5402 | 17.4 | 2.67 |
| MR122/25 | Dmt-DArg-1Nal-Ppp | C₄₄H₅₆N₈O₅ | 777.4451 | 777.4453 | 21.4 | 3.25 |
| MR115/28 | Dmt-Pro-Phe-Phe(4-Cl)-Ppp | C₄₈H₅₇CIN₆O₆ | 849.4094 | 849.4107 | 22.5 | 4.78 |
| MR123/30 | Dmt-Pro-Gly-Phe(4-Cl)-Ppp | C₄₁H₅₁CIN₆O₆ | 759.3635 | 759.3636 | 23.0 | 3.68 |
| MR127/39 | Dmt-Tic-Gly-Phe-Ppp | C₄₆H₅₄N₆O₆ | 787.4165 | 787.4184 | 24.0 | 3.91 |
| MR128/40 | Dmt-Tic-Gly-Phe(4-F)-Ppp | C₄₆H₆₃FN₆O₆ | 805.40757 | 805.4090 | 24.4 | 4.02 |
| MR129/41 | Dmt-Tic-Gly-Phe(4-Cl)-Ppp | C₄₆H₅₃CIN₆O₆ | 821.37849 | 821.3795 | 26.1 | 4.46 |
| MR232/42 | Dmt-DArg-Phe(4-Cl)Lys- | C₄₆H₆₆CIN₁₀O₆ | 889.48402 | 839.4856 | 17.0 | 2.40 |
| | Ppp | | | | | |
| MR233/43 | Dmt-DArg-Phe(4-F)-Lys | C₄₆H₆₅FN₁₀O₆ | 873.51465 | 873.5152 | 16.2 | 1.92 |
| | Ppp | | | | | |
| MR231/44 | DPhe-DPhe-DNle-DLys-Ppp | C₄₄H₆₁N₇O₅ | 768.48061 | 768.4814 | 19.9 | 3.03 |

The present invention may feature a method reducing pain without causing an opioid addiction in a subject in need thereof. In some embodiments, the method comprises administering a multifunctional compound. In other embodiments, the multifunctional compound comprises a mu opioid receptor (MOR) agonist, a delta opioid receptor (DOR) agonist, and a kappa opioid receptor (KOR) antagonist or partial agonist.

The present invention may also feature a method of modulating different opioid receptors. In some embodiments, the method comprises blocking or limiting kappa opioid receptors (KOR) and activating mu opioid receptors (MOR) and delta opioid receptors (DOR) by using a multifunctional compound on said receptors. In other embodiments, the multifunctional compound comprises a MOR agonist, a DOR agonist, and KOR antagonist or partial agonist. In further embodiments, the multifunctional compound reduces opioid side effects.

The present invention may further feature a method of blocking kappa opioid receptors (KOR) and activating mu opioid receptors (MOR) and delta opioid receptors (DOR) in a subject in need thereof. In some embodiments, the method comprises administering a multifunctional compound to the subject. In other embodiments, the multifunctional compound is a MOR agonist, a DOR agonist, and KOR antagonist or partial agonist. In further embodiments, the multifunctional compound reduces opioid side effects in the subject.

In some embodiments, opioid side effects may include but are not limited to respiratory depression, constipation, development of tolerance, and addiction.

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human under medical care or animals under veterinary care.

### Example 1

Example 1 describes non-limiting approaches to designing ORLs.

Step 1: Discover pharmacophoric structures of EM-1 and DALDA for MOR agonist/KOR antagonist activities. The C-terminus of EM-1 and DALDA may be modified with Ppp(R) (the R group may be decided by SAR results). This modification may improve their lipophilicities (a Log P increase >2) and metabolic stabilities, and thus afford high potential of BBB penetration. This modification may cause a biological profile change. The Ppp(R) group may be kept at the C-terminus, and the other positions may be modified. Substitution of Tyr with Dmt in opioid peptides can increase opioid activities dramatically, thus a Tyr residue may be replaced in both ligands with a Dmt residue or a Tmt residue, which is more sterically hindered due to an extra methyl group. EM-1 and DALDA have distinct primary structures in positions 2, 3, and 4 but a Phe residue in common. The Phe residue in both ligands may be substituted with Phe(p-X) for altering receptor selectivity and inducing KOR interactions. A Phe residue in DALDA may also be substituted with a Phe(p-X) residue to observe SAR. However, to conserve its MOR selectivity over DOR, positions 2 and 4 of DALDA may be limited to basic amino acid residues. Likewise, position 2 of EM-1 may be limited to turn making amino acid residue. A Trp residue in EM-1 may be modified with other aromatic amino acid residues.

Step 2: Build dimerized ligands of MOR agonist/KOR antagonist using pharmacophores discovered in the first step. Position 2 and 4 of EM-1 and DALDA, respectively, may be consumed. Two homo pharmacophores may be linked through a disulfide bond of homocysteine residue. Cyclic bifunctional ligands may be designed. Insertion (l, m, and/or n=1) or deletion (l, m, and/or n=0) of Bbb, Ccc, and Ddd may optimize the distance between two aromatic rings, which may be the most important factor of high potency and selectivity.

### Example 2—Multifunctional ORLs as Neuroprotectants for Ischemic Stroke Treatment

Ischemic stroke is one of the leading causes of mortality and morbidity in the world. Example 2 describes the evaluation of multifunctional ORLs, e.g., LYS436_(SEQ ID NO: 8), LYS739 (SEQ ID NO: 10) and LYS416 (YGGF-Ppp, SEQ ID NO: 37), for their neuroprotective potential using in vitro and in vivo ischemic models. In vitro, neuronal death and total reactive oxygen species level, upon exposure to hypoxia-aglycemia followed by reoxygenation or challenged with NMDA was significantly decreased when treated with non-selective opioid agonists compared to no drug treatment group. Fluorinated enkephalin-fentanyl conjugate, LYS739 (SEQ ID NO: 10) showed better neuroprotection in all in vitro ischemic models compared to biphalin. An in vivo mouse middle cerebral artery occlusion (MCAO) stroke model was utilized to screen biphalin and LYS739 (SEQ ID NO: 10). Both agonists significantly decreased brain infarct ratio and edema ration measured with TTC staining compared to saline treated group. Neuronal deficit was improved in terms of neurological score and locomotor activity with LYS739 (SEQ ID NO: 10) and biphalin treatment. All enkephalin fentanyl conjugates and biphalin demonstrated better neuroprotection compared to fentanyl treated groups. Neuroprotective effects of biphalin and multivalent analogs were reversed, in most cases, by naltrexone, a non-selective opioid antagonist. This suggests that LYS739 (SEQ ID NO: 10) is a potential neuroprotective agent for ischemic stroke.

Primary cortical neuron survival upon exposure to 3 hr H/A and 24 hr reperfusion in presence or absence of fentanyl analogs and biphalin (10 nM) was determined using MTT (see FIG. 5A) and LDH (see FIG. 5B) assays. In MTT assay, fentanyl analogs, LYS436 (57.9% more neuronal survival, p<0.0001). LYS739 (68.1% more neuronal survival, p<0.0001) and LYS416 (66.4% more neuronal survival, p<0.0001) and biphalin (42.6% more neuronal survival, p<0.001) and fentanyl (28.7% more neuronal survival, p<0.05) reproducibly improved neuronal survival compared to no drug treatment group. The protective effect of fentanyl analogs, LYS436 (p>0.05), LYS739 (p<0.01) and LYS416 (p<0.05) were significantly better than that of biphalin. They also showed better neuroprotection (LYS436: p<0.05, LYS739: p<0.0001 and LYS416: p<0.001) compared to fentanyl itself. Among the analogs. LYS739 showed the most significant activity in terms of neuronal survival. Likewise, LDH assay showed reproducible, statistically significant neuroprotection upon treatment with biphalin (30.5% less LDH release, p<0.001), LYS436 (29.37% less LDH release, p<0.001). LYS739 (45.7% less LDH release, p<0.0001). LYS416 (41.28% less LDH release, p<0.0001) and FENT (21.59% less LDH release, p<0.05) compared to no drug treated group. In comparison to biphalin, LYS739 (p<0.05) showed less neuronal death upon H/A and reoxygenation exposure. The fentanyl analogs showed less neuronal death compared to fentanyl itself. Notably, non-selective OR antagonist NTX reversed the effect of biphalin and fentanyl analogs in both assays. No statistical significant difference was found for NTX treated group compared to no drug treated group in both assays.

The effect of three fentanyl analogs, LYS436, LYS739 and LYS416 and biphalin and fentanyl (10 nM) were evaluated in primary cortical neurons exposed to 50 μM NMDA for 3 hours followed by 24 hours normal condition media exposure. Relative neuronal survival and cytotoxicity were quantified using MTT (see FIG. 6A) and LDH (see FIG. 6B) assay, respectively. MTT assay showed that LYS436 (52.1% more neuronal survival, p<0.0001), LYS739 (54.7% more neuronal survival, p<0.0001), LYS416 (43.4% more neuronal survival, p<0.001), biphalin (28.7% more neuronal survival, p<0.01) and fentanyl (22.6% more neuronal survival, p<0.05), which was statistically significant when compared to no drug treated group. Compared to biphalin, fentanyl analog LYS436 (p<0.05) and LYS739 (p<0.01) showed better neuroprotection in terms of neuronal survival quantified with MTT assay kit. These two analogs, LYS436 (p<0.01) and LYS739 (p<0.001) also increased neuronal survival when compared to fentanyl itself. Similar reproducible results were observed when the neuroprotective effect was evaluated with an LDH assay kit. With LDH assay, LYS436 (27.5% less LDH release, p<0.0001), LYS739 (28.6% less LDH release, p<0.0001), LYS416 (12.9% less LDH release, p<0.01), biphalin (18.4% less LDH release, p<0.0001) and fentanyl (10.2% less LDH release, p<0.05) showed statistically significantly increased neuroprotection compared to no drug treated group. Compared to biphalin, LYS436 (p<0.05) and LYS739 (p<0.05) treated neurons released less LDH denoting a more potent effect than biphalin. LYS436 (p<0.0001) and LYS739 (p<0.0001) also showed better neuroprotection compared to fentanyl. In both assays, non-selective OR antagonist, NTX reversed the effect of most analogs and NTX did not show any significant effect compared to the non-treated group.

Generation of total ROS in primary cortical neurons exposed to 3 hr H/A and 24 hr reoxygenation in presence or absence of OR agonist fentanyl analogs and biphalin (10 nM) was assessed in this experiment (see FIG. 7). Total ROS generation was statistically significantly reduced when neuron were treated with LYS436 (52.2% less ROS production, p<0.001), LYS739 (54.4% less ROS production, p<0.001), LYS416 (35.0% less ROS production, p<0.01) and biphalin (29.1% less ROS production, p<0.05) compared to no drug treated group. The effect of LYS739 was significantly better (p<0.05) than that of biphalin. Both LYS436 (p<0.001) and LYS739 (p<0.001) significantly decreased ROS production compared to fentanyl. Non-selective OR antagonist NTX did not show a significant decrease in ROS production compared to no drug treated group but it reversed the effect of OR agonists (except for fentanyl) used in these experiments.

As shown in FIG. 8A-C, The effect of fentanyl analog LYS739, biphalin and fentanyl on brain edema formation (see FIG. 8B) and infarct volume (FIG. 8C) after focal brain ischemia induced by 1 hr occlusion followed by 24 hr reperfusion. Compared to the vehicle treated group LYS739 produced a 59.45% reduction in edema formation, p<0.05 and biphalin produced a 56.17% reduction in edema formation, p<0.05 that was statistically significantly when administered 10 min after reperfusion at a dose of 5 mg/kg in saline (i.p.). Fentanyl (0.2 mg/kg, 10 minute post reperfusion) and/or antagonist NTX (1 mg/kg, 10 min before stroke) did not show any significant reduction in edema formation compared to saline treated group. LYS739 produced a 67.7-% reduction in infarct ratio, p<0.0001) and biphalin produced a 67.0% reduction in infarct ratio, p<0.0001 that were statistically significant compared to saline treated group. Again, fentanyl and NTX did not show any improvement in terms of infarct ratio. For both edema formation and infarction volume, NTX reversed the effect of both LYS739 and biphalin. Mean cerebral blood flow reduction ±SEM in ischemic brain for saline group 80.7±1.2%, BIP 81.1±1.3%, BIP+NTX 79.7±2.0%, LYS739 82.1±1.2%. LYS739+NTX 80.6±2.2%, FENT 80.4±1.6%, NTX 76.9±2.0%.

Twenty-four hours after the reperfusion neurological score was evaluated in the experimental groups (see FIG. 9). LYS739 (30.4% improvement, p<0.05) and biphalin (25.5% improvement, p<0.05) significantly improved the neurological score compared to saline treated control group. Fentanyl or OR antagonist NTX did not improve any neurological score under the same experimental conditions. NTX reversed the effect of both LYS739 and biphalin but the effects were not statistically significant.

Locomotor activity (horizontal activity, vertical activity, total distance, rest time, stereotype counts and number of movements) was evaluated 24 hr after reperfusion in experimental animals (Table 8). Before the start of surgery all animals went through locomotor evaluation to get the baseline. Both LYS739 and biphalin (5 mg/kg. 10 min post reperfusion, i.p.) statistically significantly improved all the locomotor parameters compared to saline treated control animals. When compared the effect of LYS739 to that of biphalin most of the parameter were improved although they were not statistically significant except for vertical activity (p<0.05). But, in comparison to fentanyl treated group, both LYS739 and biphalin showed better locomotor activity and the effects were statistically significant. Non-selective OR antagonist NTX did not improve any locomotor parameters.

Table 8 shows measurement of locomotor activity 24 h after stroke and drug treatments. Data represent the mean±S.E.M. of 4-5 independent determinations: numbers indicated in parenthesis in the line of the table columns donate to the number of experimental animals per group. ‘*’ Compared to Saline treated group—*p<0.05; **p<0.01: ***p<0.001; ****p<0.0001; ‘#’ Compared to biphalin treated group—#p<0.05; ##p<0.01; ###p<0.001; ####p<0.0001: ‘Φ’ Compared to fentanyl treated group—Φ p<0.05; ΦΦ p<0.01; ΦΦΦ p<0.001; ΦΦΦΦ p<0.0001

TABLE 8

| | | 0.9% | | BIP + | | LYS739 + | | |
|---|---|---|---|---|---|---|---|---|
| | Sham | Saline | BIP | NTX | LYS739 | NTX | FENT | NTX |
| Parameters | (N = 7) | (N = 7) | (N = 7) | (N = 5) | (N = 4) | (N = 4) | (N = 4) | (N = 4) |
| Horizontal | 1300 ± | 150 ± | 760 ± | 390 ± | 1000 ± | 120 ± | 140 ± | 160 ± |
| Activity | 230 **** # | 34 ## | 75 ** ϕ | 130 | 160 *** ϕϕϕ | 9 ## | 50 # # | 36 # |
| Vertical | 36 ± | 0 ## ϕ | 28 ± | 20 ± | 50 ± | 0 ## | 3 ± | 1.8 ± |
| Activity | 8 ** | | 3 ** | 2 | 13 **** # ϕϕϕϕ | | 3 # | 1 ## |
| Total | 1100 ± | 17 ± | 650 ± | 88 ± | 680 ± | 8 ± | 15 ± | 45 ± |
| Distance | 280 **** | 5 # | 81 ** ϕϕ | 64 # | 110 ** ϕϕ | 2 ## | 6 ## | 16 # |
| (CM) | | | | | | | | |
| No. of | 75 ± | 10 ± | 42 ± | 19 ± | 61 ± | 6 ± | 6 ± | 11 ± |
| Movements | 16 ▪▪▪▪▪▪▪ # | 2 # | 6 * ϕ | 6 | 7 ** ϕϕ | 2 # | 1 # | 3 |
| Stereotpy | 970 ± | 62 ± | 390 ± | 230 ± | 340 ± | 39 ± | 83 ± | 77 ± |
| | 110 | 12 | 46 | 71 | 76 | 2 | 35 | 13 |
| Counts | **** #### | ## | ** ϕ | | * | ## | # | # |
| Rest Time | 220 ± | 300 ± | 240 ± | 270 ± | 220 ± | 300 ± | 300 ± | 300 ± |
| (Seconds) | 19 **** | 1 ## | 7 ** ϕϕϕ | 7 | 14 **** ϕϕϕϕ | 27 ### | 1 ### | 2 ## |

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method reducing pain without causing an opioid addiction in a subject in need thereof, the method comprising: administering a multifunctional compound, wherein said multifunctional compound is a mu opioid receptor (MOR) agonist, a delta opioid receptor (DOR) agonist, and a kappa opioid receptor (KOR) antagonist or partial agonist.

2. The method of claim 1, wherein the multifunctional compound is selected from a group consisting of: SEQ ID NO: 11, SEQ ID NO: 12, SEQ ID NO: 13, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 37, SEQ ID NO: 39, SEQ ID NO: 40, SEQ ID NO: 41, SEQ ID NO: 42, SEQ ID NO: 43, and SEQ ID NO: 44.

3. The method of claim 1, wherein the multifunction compound is SEQ ID NO: 18.

4. The method of claim 1, wherein the multifunctional compound is neuroprotective.

5. The method of claim 1, wherein the pain is chronic pain or migraine.

6. A method of modulating different opioid receptors, the method comprising blocking or limiting kappa opioid receptors (KOR) and activating mu opioid receptors (MOR) and delta opioid receptors (DOR) by using a multifunctional compound on said receptors, wherein said multifunctional compound is a MOR agonist, a DOR agonist, and KOR antagonist or partial agonist; wherein the multifunctional compound reduces opioid side effects.

7. The method of claim 6, wherein the multifunctional compound is selected from a group consisting of: SEQ ID NO: 11, SEQ ID NO: 12, SEQ ID NO: 13, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 37, SEQ ID NO: 39, SEQ ID NO: 40, SEQ ID NO: 41, SEQ ID NO: 42, SEQ ID NO: 43, and SEQ ID NO: 44.

8. The method of claim 6, wherein the multifunction compound is SEQ ID NO: 18

9. The method of claim 6, wherein the multifunctional compound is neuroprotective.

10. A method of blocking kappa opioid receptors (KOR) and activating mu opioid receptors (MOR) and delta opioid receptors (DOR) in a subject in need thereof, the method comprising: administering a multifunctional compound to the subject, wherein said multifunctional compound is a MOR agonist, a DOR agonist, and KOR antagonist or partial agonist; wherein the multifunctional compound reduces opioid side effects in the subject.

11. The method of claim 10, wherein the multifunctional compound is selected from a group consisting of: SEQ ID NO: 11, SEQ ID NO: 12, SEQ ID NO: 13, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 37, SEQ ID NO: 39, SEQ ID NO: 40, SEQ ID NO: 41, SEQ ID NO: 42, SEQ ID NO: 43, and SEQ ID NO: 44.

12. The method of claim 10, wherein the multifunction compound is SEQ ID NO: 18.

13. The method of claim 10, wherein the multifunctional compound is neuroprotective.
